(19) United States
(12) Patent Application Publication
(10) Pub. No.: US 2020/0261489 A1
(43) Pub. Date: Aug. 20, 2020

(54) HIGH CONCENTRATION FORMULATIONS

(71) Applicant: JAZZ PHARMACEUTICALS IRELAND LIMITED, Dublin (IE)

(72) Inventors: Mariana Dimitrova, Wallingford, PA (US); William J. Bennett, Turnersville, NJ (US); Qi Wang, Pennington, NJ (US)

(21) Appl. No.: 16/105,319

(22) Filed: Aug. 20, 2018

Related U.S. Application Data
(63) Continuation of application No. PCT/US2018/045152, filed on Aug. 3, 2018.
(60) Provisional application No. 62/540,657, filed on Aug. 3, 2017.

(51) Int. Cl. A61K 31/7088

(52) U.S. Cl. CPC A61K 31/7088; A61K 9/0019

(57) ABSTRACT

Low-viscosity, high concentration nucleic acid compositions that can be administered by multiple parenteral routes may allow for less frequent dosing than nucleic acid products currently on the market. In particular, low-viscosity defibrotide formulations for subcutaneous, intramuscular, and intraperitoneal administration are more convenient to the patient and/or are administered outside of the hospital setting. Formulations of the invention may be used for the treatment of numerous conditions including for example, treatment of peripheral arteriopathies, treatment of acute renal insufficiency, treatment of acute myocardial ischemia, and treatment and prevention of sinusoidal obstruction syndrome or VOD.

### 1. CROSS REFERENCE TO RELATED APPLICATIONS

This application is a continuation of PCT/US2018/045152 filed Aug. 3, 2018, which claims the benefit of priority to U.S. Provisional Application No. 62/540,657, filed Aug. 3, 2017 the contents of each of which are incorporated by reference in their entireties.

### 2. BACKGROUND OF THE INVENTION

Defibrotide, a nucleic acid salt, is a complex mixture of random sequence, predominantly single-stranded polydeoxyribonucleotides derived from animal mucosal DNA. It has protective effects on vascular endothelial cells, particularly those of small vessels and has antithrombotic, anti-inflammatory and antiischemic properties.

The sodium salt of defibrotide is commercially sold as Defitelio® (Gentium S.r.L., Villa Guardia, Italy) and is currently approved for the treatment of adult and pediatric patients with hepatic veno-occlusive disease (VOD), also known as sinusoidal obstruction syndrome (SOS), with renal or pulmonary dysfunction following hematopoietic stem-cell transplantation (HSCT). It is administered to patients by 2-hour intravenous infusions every 6 hours for a minimum of 21 days. The frequency and large volumes of the infusion regimen requires that patients have a second IV line for defibrotide administration to avoid mixing defibrotide with other drugs that must be given IV. The treatment regimen would not be compatible in an outpatient dosing for additional disease indications for which defibrotide may be shown to be therapeutic. Therefore, it would be beneficial to administer defibrotide in a way that is more convenient to the patient to allow dosing in an outpatient setting, allow patients to self-administer at home via a compatible administration device, or reduce dosing duration and liquid volume in a hospital setting. Thus there is a need for new formulations of defibrotide which would permit new and more patient convenient dosing regimens for administration of pharmaceutically effective doses at home.

### 3. SUMMARY OF THE INVENTION

This invention covers a broad range of nucleic acids and their salts, including defibrotide, and the ability to make high concentration formulations of these molecules while keeping the viscosity and osmolality at physiologically relevant levels. These high concentration formulations offer numerous benefits to the patient, including for example, the ability to be administered subcutaneously and to be administered by the patient outside of a hospital setting. The advantages of self-administration and administration by other than the IV route are felt by the patient and their families as well as by the hospital. The amount of time and resources that the hospital needs to treat and monitor these patients are significantly reduced which provides a reduced economic burden on both the hospital and the patient. The formulations provided herein are specifically related to defibrotide; however, it is understood that the invention applies to a broad range of nucleotide products, for example, single and double-stranded DNA or RNA products, such as DNA and RNA vaccines.

Provided herein are nucleic acid compositions for therapeutic administration which may be administered by multiple parenteral routes and which may improve the quality of life for patients by less frequent and/or shorter duration of dosing than similar nucleic acid products currently on the market. More particularly, provided are low-viscosity, high concentration nucleic acid formulations that can also be administered by routes other than intravenous, including for example, subcutaneous, intramuscular, and/or intraperitoneal routes. In certain embodiments, high concentration nucleic acid formulations are self-administered and/or administrated in an out-patient basis. In specific embodiments, the nucleic acid is defibrotide. Formulations of the invention may be used for the treatment and/or prevention of numerous conditions including, for example, Hematopoietic Stem Cell Transplantation (HSCT) related complications such as sinusoidal obstruction syndrome or hepatic veno-occlusive disease (VOD), Graft versus Host Disease (GvHD), Transplant-Associated Thrombotic Microangiopathy (TA-TMA) or Idiopathic Pneumonia Syndrome. Other conditions including, for example, other TMAs including Thrombotic Thrombocytopenic Purpura (TTP) and Hemolytic-Uremic Syndrome (HUS), Acute Myocardial Ischemia, Ischemic Stroke, Ischemia Reperfusion Injury in solid organ transplantation, Acute Respiratory Distress Syndrome (ARDS), Sickle Cell Vaso-occlusive Crisis (VOC) Sickle Cell Related Acute Chest Syndrome, Disseminated Intravascular Coagulation (DIC), Sepsis, Renal Insufficiency, other Coronary or Peripheral Artery Diseases, Hematological Malignancies or Solid Tumors.

In some aspects, the present disclosure provides low-viscosity pharmaceutical formulations comprising a nucleic acid at a concentration of at least 80 mg/mL. In some embodiments, the nucleic acid concentration is between about 85 mg/mL and about 400 mg/mL. In some embodiments, the viscosity of the formulation is: a) less than about 70 cP; b) between about 5 cP and 65 cP; or c) between about 10 cP and about 65 cP. In some embodiments, the viscosity is measured: a) at room temperature; b) between about 15° C. and about 35° C.; or c) between about 21° C. and about 23° C.

In some embodiments, the low-viscosity pharmaceutical formulation further comprises glycylglycine. In some embodiments, the glycylglycine concentration is a) between about 5 mM and about 100 mM; b) between about 5 mM and 60 mM; or c) between about 10 mM and about 40 mM

In some embodiments, the low-viscosity pharmaceutical formulation has an osmolality of a) between about 240 mOsm/kg and about 600 mOsm/kg; or b) between about 300 mOsm/kg and about 550 mOsm/kg.

In some embodiments, the nucleic acid in the low-viscosity pharmaceutical formulation comprises polynucleotide or oligonucleotides of ribonucleic acid or deoxyribonucleic acid. In some embodiments, the molecular weight of the nucleic acid is a) between about 5,000 to about 50,000 daltons; b) between about 13,000 to about 30,000 daltons; or c) between about 16,000 to about 20,000 daltons.

In some embodiments, the nucleic acid comprises polydisperse, random sequences. In some embodiments, the nucleic acid is present as predominantly single-stranded polydeoxyribonucleotides.

In some embodiments, the low-viscosity pharmaceutical formulation comprises single-stranded polydeoxyribonucleotides that are random sequences that correspond to the following formula:

P1-5,(dAp)12-24,(dGp)10-20,(dTp)13-26,(dCp)10-20

wherein: P=phosphoric radical
dAp=deoxyadenylic monomer
dGp=deoxyguanylic monomer
dTp=deoxythymidylic monomer
dCp=deoxycytidylic monomer

In some embodiments, the low-viscosity pharmaceutical formulation comprises a buffer or excipient selected from sodium citrate, sodium succinate, histidine, TRIS buffer, HEPES buffer, sodium chloride, arginine, lidocaine, and/or polysorbate-80. In some embodiments, the low-viscosity formulation comprises a buffer or excipient so that the nucleic acid is in the form of an alkali metal salt. In some embodiments, the buffer or excipient includes a sodium salt. In some embodiments, the buffer or excipient is sodium citrate, sodium succinate, or sodium chloride. In some embodiments, the buffer or excipient is sodium citrate, sodium succinate, or sodium chloride at a concentration of less than about 80 mM sodium salt.

In some embodiments, the buffer or excipient is sodium citrate at a concentration of between 20-34 mM.

In some aspects, the present disclosure provides low-viscosity pharmaceutical formulations comprising between 85 mg/mL to about 400 mg/mL of a composition comprising over 70% single-stranded, polydisperse polydeoxyribonucleotides, wherein each polydeoxribonucleotide comprises between 45 and 65 bases and has a mean molecular weight between 13 kDa and 20 kDa, and glycylglycine at a concentration of between about 5 mM and about 100 mM.

In some aspects, the present disclosure provides low-viscosity pharmaceutical formulations comprising between 150 mg/mL to about 250 mg/mL of a nucleic acid composition comprising a nucleic acid over 70% single-stranded, polydisperse polydeoxyribonucleotides, wherein each polydeoxribonucleotide comprises between 45 and 65 bases and has a mean molecular weight between 13 kDa and 20 kDa, and glycylglycine at a concentration of between about 5 mM and about 60 mM, wherein the formulation has a viscosity between about 5 and about 70 cP when measured at between 15° C. and 25° C., and an osmolality between about 300 mOsm/kg and 550 mOsm/kg, and wherein the formulation is formulated for parenteral administration to a patient.

In some aspects, the present disclosure provides low-viscosity pharmaceutical formulations comprising between 100 mg/mL to about 400 mg/mL of defibrotide, and glycylglycine at a concentration of between about 5 mM and about 60 mM, wherein the formulation has a viscosity between about 5 and about 60 cP when measured at between 15° C. and 25° C., and an osmolality between about 240 mOsm/kg and 700 mOsm/kg, and wherein the formulation is formulated for parenteral administration to a patient.

In some embodiments, the viscosity in the low-viscosity pharmaceutical formulation decreases over time. In some embodiments, the viscosity decreases during storage. In some embodiments, the viscosity decreases under increasing shear, agitation, and/or pressure. In some embodiments, the shear increases during administration of the pharmaceutical formulation. In some embodiments, the shear increases during administration of the pharmaceutical formulation via a needle or device.

In some embodiments, the low-viscosity pharmaceutical formulation is formulated for subcutaneous, intramuscular, or intraperitoneal administration. In some embodiments, the formulation demonstrates extended systemic half-life compared to a formulation delivered via intravenous administration. In some embodiments, the subcutaneously-delivered formulation exhibits lower peak-to-trough ratios of plasma concentrations compared to a formulation delivered via intravenous administration. In some embodiments, the subcutaneously-delivered formulation exhibits improves efficacy and/or an improved safety profile compared to a formulation delivered via intravenous administration.

In some embodiments, the low-viscosity pharmaceutical formulation isotonic or thixotropic.

In some embodiments, the low-viscosity pharmaceutical formulation may be self-administered by a patient.

In some aspects, the present disclosure provides a device for subcutaneous administration of low-viscosity formulations comprising a nucleic acid at a concentration of at least 80 mg/mL.

In some aspects, the present disclosure provides methods of treating a disease comprising administering the low-viscosity formulation of any of claims 1-33, wherein the disease is selected from thrombosis, Hematopoietic Stem Cell Transplantation (HSCT) related complications including sinusoidal obstruction syndrome or hepatic veno-occlusive disease (VOD), Graft versus Host Disease (GvHD), Transplant-Associated Thrombotic Microangiopathy (TA-TMA) or Idiopathic Pneumonia Syndrome, other TMAs including Thrombotic Thrombocytopenic Purpura (TTP) and Hemolytic-Uremic Syndrome (HUS), Acute Myocardial Ischemia, Ischemic Stroke, Ischemia Reperfusion Injury in solid organ transplantation, Acute Respiratory Distress Syndrome (ARDS), Sickle Cell Vaso-occlusive Crisis (VOC), Sickle Cell Related Acute Chest Syndrome, Disseminated Intravascular Coagulation (DIC), Sepsis, Renal Insufficiency, other Coronary or Peripheral Artery Diseases, Hematological Malignancies or Solid Tumors.

In some embodiments, the low-viscosity formulation is administered at a dosing regimen that provides improved patient quality of life by requiring a reduced administration volume and/or allowing less-frequent administration.

Thus in one embodiment, provided is a low-viscosity formulation for therapeutic administration to a patient, comprising a nucleic acid; wherein the nucleic acid is present in a concentration of at least 80 mg/mL. In some embodiments, the nucleic acid is present in a concentration between 85 and 400 mg/mL. In some embodiments, the nucleic acid is present in a concentration that is at least 85, 90, 95, or 100 mg/mL. The nucleic acid can be present in a concentration between 100 and 400 mg/mL, or 100 and 300 mg/mL. In some embodiments, the nucleic acid has between 45 and 65 bases and/or a mean molecular weight between 13 and 20 kDa. In certain embodiments, the nucleic acid is predominantly single stranded. Thus preferably, the nucleic acid is at least 70%, 75%, 80%, 85%, 90%, or 95% single stranded. In some embodiments, up 5%, 10%, 15%, 20%, 25%, or up to 30% of the bases in the nucleic acid are paired. In other embodiments, the nucleic acid is up 5%, 10%, 15%, 20%, 25%, or up to 30% double stranded.

In some embodiments, the nucleic acid is present as an alkali metal salt. In certain embodiments, the alkali metal salt is a sodium salt. In specific embodiments, the nucleic acid is predominantly single stranded polydeoxyribonucleotides. In some preferred embodiments, the nucleic acid is predominantly single stranded polydeoxyribonucleic sodium salts. In specific embodiments, the nucleic acid is defibrotide.

For improved patient convenience it is important for injectables to be administered to patients as low-viscosity, isotonic, and/or thixotropic therapeutic formulations. In the case of defibrotide, as the concentration is increased a very small change in concentration results in a large change in viscosity and this variation is further affected by temperature. The current invention allows the concentration to be increased while still meeting the criteria for well-tolerated injectable biologics.

Thus, in one embodiment, the above formulations have a viscosity that is less than 70 centipoise (cP). In one embodiment, the viscosity is between 5 and 65 cP, or 10 and 60 cP. Preferably the viscosity is measured under room temperature conditions, such as from 15° C. to 35° C. More preferably, the viscosity is measured between 18° C. to 25° C. Even more preferably, the viscosity is measured at between 21° C. to 23° C.

In another embodiment, the above formulations have an osmolality of between 240 and 700 mOsm/kg. In other embodiments, the above formulations have an osmolality of between 300 and 500 mOsm/kg. In specific embodiments, the above formulations have a pH between 6.8 and 8.5 or between 7 and 8.

Certain buffers or excipients may be used to control the stability, viscosity and/or osmolality. In one embodiment, the above formulations comprise one or more buffers or excipients. In certain embodiments, the excipient is selected from the group consisting of sodium citrate, succinate, sodium chloride, arginine, lysine, lidocaine, or polysorbate-80 (“PS-80”). In some embodiments, the buffer is selected from the group consisting of glycylglycine, histidine, tris(hydroxymethyl)aminomethane (“TRIS”), sodium citrate, or 4-(2-hydroxyethyl)-1-piperazineethanesulfonic acid (“HEPES”) buffer. In one preferred embodiment, the buffer is a dipeptide, such as for example L-Carnosine or glycylglycine. Glycylglycine alone and in and combinations with other excipients improves the solution properties of the formulation by minimizing viscosity and/or osmolality for a given concentration of nucleic acid. Glycylglycine containing formulations manifest solution attributes best optimized to physiologically relevant conditions known to improve tolerability and minimize discomfort upon injection. Thus, in one embodiment, provided is a low-viscosity formulation for therapeutic administration to a patient, comprising a nucleic acid; wherein the nucleic acid is present in a concentration of at least 80 mg/mL; and glycylglycine. In some preferred embodiments, the nucleic acid is defibrotide. Defibrotide manifests non-Newtonian shear thinning and thixotropic behavior in liquid formulations, and this behavior is prominently evident in high concentration liquid formulations. Thus, in certain embodiments, provided is a low-viscosity formulation for therapeutic administration to a patient, comprising at least 80 mg/mL of a solution of defibrotide; and glycylglycine. In some embodiments, glycylglycine is present in an amount between 5 and 100 mM. More preferably, glycylglycine is present in an amount between 5 and 60 mM or 10 and 40 mM.

In another embodiment, provided is a low-viscosity formulation for therapeutic administration to a patient, comprising: between 100 and 300 mg/mL of a nucleic acid which contains greater than 70% single stranded, polydisperse polydeoxyribonucleotides having between 45 and 65 bases and a mean molecular weight between 13 and 20 kDa; and an excipient comprising glycylglycine in an amount between 10 and 60 mM. In yet another embodiment, provided is a low-viscosity formulation for therapeutic administration to a patient, comprising: between 150 and 250 mg/mL of a nucleic acid which contains greater than 70% single stranded, polydisperse polydeoxyribonucleotides having a mean length between 45 and 65 bases and a mean molecular weight between 13 and 20 kDa; an excipient comprising glycylglycine in an amount between 10 and 100 mM; and wherein the formulation has a viscosity between 5 and 70 cP, and/or an osmolality of between 240 and 550 mOsm/kg and is suitable for parenteral administration to a patient. In some preferred embodiments, the nucleic acid is defibrotide.

In one embodiment, provided is a low-viscosity formulation for therapeutic administration to a patient, comprising: between 100 and 300 mg defibrotide/mL, comprising greater than 70% single stranded, polydisperse polydeoxyribonucleotides having a mean length between 45 and 65 bases and a mean molecular weight between 13 and 20 kDa; an excipient comprising glycylglycine in an amount between 10 and 100 mM; wherein the formulation has a viscosity between 5 and 70 cP, an osmolality of between 240 and 500 mOsm/kg and is suitable for parenteral administration to a patient.

In one embodiment, provided is a method of parenterally administering a low-viscosity formulation of the invention. In some embodiments, the formulation is suitable for subcutaneous administration. In certain embodiments, the formulations comprise a device for subcutaneous delivery including self-administration. In a preferred embodiment, provided is a method of delivering subcutaneously a dose of defibrotide over 5 minutes to 3 hours in between 5 and 50 mL of aqueous fluid.

In other aspects, provided herein are methods of making the formulations disclosed herein. In additional aspects, provided are methods of packaging a formulation of the invention. In certain aspects, provided are methods of packaging a formulation of the invention in a device that is capable of subcutaneous administration.

In one embodiment, the above formulations can be used for self-administration by patients. In certain embodiments, the above formulations can be used for administration outside of a hospital setting.

In some embodiments, the condition or disease is hepatic VOD with renal or pulmonary dysfunction following hematopoietic stem-cell transplantation.

### 4. BRIEF DESCRIPTION OF THE FIGURES

FIG. 1A is a graph showing the viscosity of various formulations as a function of defibrotide concentration using 3 different formulation buffers: sodium citrate (diamonds), glycylglycine (squares) or a mixture of sodium citrate and glycylglycine (triangles).

FIG. 1B is a graph showing the viscosity as a function of temperature of formulations containing sodium citrate (blue diamonds), GlyGly (red squares), or GlyGly and sodium citrate (green triangles).

FIG. 1C is a graph showing viscosity decrease over time in formulations containing 20 mM GlyGly (blue circles), 20 mM GlyGly and 34 mM sodium citrate (orange squares), 20 mM GlyGly and 100 mM sodium succinate (blue triangles) and 20 mM GlyGly and 20 mM sodium chloride (red diamonds).

FIG. 1D is a graph showing the osmolality of various formulations as a function of defibrotide concentration using either sodium citrate (diamonds) or glycylglycine (squares).

FIG. 2A is a graph showing the viscosity of 200 mg/mL defibrotide formulations in the presence of various buffers or excipients.

FIG. 2B is a graph showing the osmolality of 200 mg/mL defibrotide formulations in the presence of various buffers or excipients.

FIG. 3A is a graph showing the osmolality increase as a function of sodium salts.

FIG. 3B is a graph showing the viscosity over time of 180 mg/mL defibrotide formulations in the presence of glycylglycine buffers and sodium citrate solutions (containing 0, 20, 34, 80, or 100 mM sodium citrate).

FIG. 4 is a graph showing the effects of temperature over time on the viscosity of 200 mg/mL defibrotide formulations containing glycylglycine buffer.

FIG. 5A is a graph showing the effects of temperature over time on the osmolality of 200 mg/mL defibrotide formulations containing citrate buffer.

FIG. 5B is a graph showing the effects of temperature over time on the osmolality of 200 mg/mL defibrotide formulations containing glycylglycine buffer.

FIG. 6 is a graph showing the pharmacokinetics of three different 200 mg/mL defibrotide formulations of the invention administered subcutaneously using an animal model in comparison to subcutaneous and intravenous administration of commercially available Defitelio®.

FIG. 7 is a graph showing simulated pharmacokinetic profiles of defibrotide following 4× daily 2-hour infusions of 6.25 mg/kg and 2× daily subcutaneous administration of 18 mg/kg assuming 70% bioavailability.

### 5. DETAILED DESCRIPTION OF THE INVENTION

Defibrotide (CAS number 83712-60-1) is a substance derived from materials of natural origin. It is the sodium salt of relatively low molecular weight polydeoxyribonucleotides which are obtained by extraction from animal mucosa. Defibrotide has a diverse size range and is known to have a mean molecular weight (MW) between 13 and 20 kDa. Defibrotide can be obtained according to U.S. Pat. Nos. 4,985,552 and 5,223,609 and/or presents the physical/chemical characteristics described in the same U.S. Pat. Nos. 4,985,552 and 5,223,609, each of which is incorporated herein by reference. Synthetic defibrotide, presented as phosphodiester oligonucleotides that mimic the therapeutic action of defibrotide are described in US20110092576 which is incorporated herein by reference in its entirety.

Defibrotide has numerous therapeutic applications, including use as an anti-thrombotic agent (U.S. Pat. No. 3,829,567), treatment of peripheral arteriopathies, treatment of acute renal insufficiency (U.S. Pat. No. 4,694,134), and treatment of acute myocardial ischaemia (U.S. Pat. No. 4,693,995). More recently, defibrotide has been used for the treatment and prevention of sinusoidal obstruction syndrome/veno occlusive disease (EU clinical trial EudraCT:2004-000592-33, US clinical trial 2005-01 (ClinicalTrials.gov identifier: NCT00358501). Patients are treated with a 6.25 mg/kg dose given as a two hour intravenous infusion every six hours until signs and symptoms of VOD are mitigated. As mentioned above, Defibrotide is currently sold under the name Defitelio® as a single vial for injection (commercially available from Gentium S.r.L., Villa Guardia, Italy; see package insert available at dailymed.nlm.nih.gov/dailymed/search.cfm?labeltype=all&query=defibrotide). Defitelio® is prepared as an intravenous infusion by a dilution in 5% Dextrose Injection, USP or 0.9% Sodium Chloride Injection, USP. Intravenous preparation is used within 4 hours if stored at room temperature or within 24 hours if stored under refrigeration. It is administered for a total of 8 hours over 4 intravenous infusions.

The development of novel defibrotide formulations and/or dosage forms for administration by intravenous (IV), subcutaneous (SC), intramuscular (IM), or oral (PO) routes of administration may offer improved quality of life for the patients undergoing treatment. For example, decreasing the frequency from 4 times daily to once or twice daily as well as decreasing the duration of the infusions may offer quality of life improvements to patients while being treated. SC route of administration of defibrotide may offer significant reduction of the time for clinical administration and enable outpatient dosing of the product for as long as needed. Combination products including large volume SC delivery devices can also offer added convenience and faster administration by health-care professionals (HCP), care-givers or even self-administration by the patients. The oral route of administration may be associated with ease of dose preparation and administration, reduced pain and is often preferred by patients. The examples of formulation, drug delivery and dosage forms development studies listed above, focus on improving quality of life and patients' experience while on treatment with defibrotide.

In some embodiments, the route of administration affects the efficacy and/or longevity of the formulations of the present disclosure. In some embodiments, subcutaneous, intramuscular and/or intraperitoneal administration is associated with an extended systemic half-life compared to the same formulation administered intravenously. In some embodiments, subcutaneous administration of the formulation provides lower peak-to-trough ratios of plasma concentrations compared to the same formulation administered intravenously. In some embodiments, subcutaneous administration provides improved efficacy and/or improves the safety profile of the formulation compared to the same formulation administrated intravenously.

### 5.1 Definitions

The following definitions are given for a better understanding of the present invention:

As used herein, the term “nucleic acid” includes “nucleic acids and their salts” and refers to molecules which are comprised of nucleotides, including polymers or large biomolecules composed of nucleotide units linked together in a chain; this includes polynucleotides and oligonucleotides including those comprised of ribose and/or deoxyribose monomers; they can be uniform in size and/or sequence or they can be polydisperse; they can be of any length, including a mixture of different lengths, but some embodiments are generally between 10-400 bases, 20-200 bases, or 45-60 bases long; in some embodiments the mean MW is between 5 and 50 kilodaltons (“kDa), between 13 and 30 kDa, or between 13 and 20 kDa, or between 16 to 20 kDa; they can be single or double stranded, but some embodiments are mostly single stranded polydeoxyribonucleotide salts within the limits stated elsewhere in this application. This also includes DNA sequences that are obtained from the controlled depolymerization of animal intestinal mucosal genomic DNA and, as one embodiment, includes defibrotide.

As used herein, the term “defibrotide” refers to both natural and synthetic sources of defibrotide, including synthetic phosphodiester oligonucleotides as described in US patent application number 20110092576. The term defibrotide identifies a polydeoxyribonucleotide that is obtained by extraction from animal and/or vegetable tissues but which may also be produced synthetically; the polydeoxyribonucleotide is normally used in the form of an alkali-metal salt, generally a sodium salt, and generally has a molecular weight of 13 to 30 kDa (CAS Registry Number: 83712-60-1). Preferably, defibrotide is obtained according to U.S. Pat. Nos. 4,985,552 and 5,223,609 and/or presents the physical/chemical characteristics described in the same U.S. Pat. Nos. 4,985,552 and 5,223,609, herein incorporated by reference. More in particular, defibrotide is a mixture of polydeoxyribonucleotides having formula of random sequence: P1-5, (dAP)₁₂₋₂₄, (dGP)₁₀₋₂₀, (dPp)₁₃₋₂₆, (dCP)₁₀₋₂₀, where: P=phosphoric radical; dAp=deoxyadenylic monomer; dGp=deoxyguanylic monomer; dTp=deoxythymidinic monomer; dCp=deoxycytidynic monomer; and/or shows the following chemical/physical characteristics: electrophoresis=homogeneous anodic mobility, and/or extinction coefficient, E₁ cm^{1%} at 260±1 nm nm=220±10, and/or E₂₃₀/E₂₆₀=0.45±0.04, and/or coefficient of molar extinction (referred to phosphorous) ε(P)=7.750±500, and/or rotatory power [α]D^{20°}=53°±6; and/or reversible hyperchromicity, indicated as % in native DNA and/or h=15±5.

As used herein, the term “polydeoxyribonucleotide” refers to a polymer whose constituent monomer is a deoxyribonucleotide.

As used herein, the term “oligodeoxyribonucleotide” refers to any oligonucleotide composed of deoxyribose monomers.

As used herein, the term “mean MW” refers to the mean or average molecular weight of the polymer.

The term, “glycylglycine” or “Gly-Gly” or “GlyGly” or “glygly” as used herein, refers to a simple peptide, made of two glycine molecules (glycine is a simple, nonessential amino acid); the dipeptide is used in the synthesis of more complicated peptides. Glycylglycine, an ampholyte, is also sometimes referred to as Diglycine, Diglycocoll, Glycine dipeptide, N-Glycylglycine. It can be made by methods such as those described in CN patent application 101759767 which is incorporated herein by reference in its entirety.

The term, “excipient,” as used herein, refers to any substance that may be formulated with defibrotide and may be included for the purpose of enhancement of the defibrotide in the final dosage form, such as facilitating its bioavailability, reducing viscosity and/or osmolality, enhancing solubility of the composition or to enhance long-term stability. Excipients can also be useful in the manufacturing process, to aid in the handling of the active substance. The selection of appropriate excipients also depends upon the route of administration and the dosage form, as well as the active ingredient and other factors. Accordingly, defibrotide may be combined with any excipient(s) known in the art that allows tailoring its performance during manufacturing or administration as well as its in vitro and in vivo performance. Many of these excipients may be utilized to tailor the pharmacokinetic profiles of defibrotide formulations.

The term, “buffer” or “buffering agent,” as used herein, refers to a solution which resists changes in the hydrogen ion concentration on the addition of a small amount of add or base. This includes, for example, a weak acid or base that is used to maintain the pH of a solution near a chosen pH value after the addition of another acidic or basic compound. The function of such buffer or buffering agent is to prevent a change in pH of a solution when acids or bases are added to said solution.

The term, “pH adjusting agent,” as used herein, refers to an acid or base used to alter the pH of a solution to a chosen pH value. The function of such an agent is to alter the pH of a solution to the desired value subsequent to the addition of acidic or basic compounds.

The term, “formulation,” as used herein, refers to compositions for therapeutic use, including, for example, a stable and pharmaceutically acceptable preparation of a pharmaceutical composition or formulation disclosed herein.

The term, “low-viscosity formulation,” as used herein, refers to a formulation which has a viscosity that is less than about 70 centipoise (cP). Normally viscosity is measured at ambient/room temperatures of (e.g. 15° C. to 35° C.; between 18° C. to 25° C. or between 21° C. to 23° C.) depending on the geographic region and/or weather conditions of the room in which it is being measured.

The term, “aqueous formulation,” as used herein, refers to a water-based formulation, in particular, a formulation that is an aqueous solution.

The term, “high concentration formulation” or “high concentration liquid formulation” or “HCLF” as used herein, refers to those formulations where the concentration of the nucleic acid is about 80 mg/mL or higher; or about 85 mg/mL or higher.

The term, “high concentration defibrotide formulations” as used herein, refers to those formulations where the defibrotide concentration is about 80 mg/mL or higher.

The term, “pharmacokinetic” or “PK” as used herein, refers to in vivo movement of an individual agent in the body, including the plasma concentration time profiles and kinetic parameters like the maximum concentration (Cmax), area under the curve (AUC), and time to maximum concentration of said agent (Tmax).

The phrase “pharmaceutically acceptable” or “acceptable”, as used in connection with compositions of the invention, refers to molecular entities and other ingredients of such compositions that are physiologically tolerable and do not typically produce untoward reactions when administered to an animal and/or human. Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “physiologically relevant” as used herein, refers to a measurement, level or amount that is suitable for use in a pharmaceutical, therapeutic or other dosage form to be administered to an animal subject, particularly a human subject.

As used herein, the term “parenteral” refers to any non-oral means of administration. It includes intravenous (i.v. or IV) infusion, IV bolus injection, subcutaneous (s.c. or SC) and intramuscular (i.m. or IM) injection.

As used herein, the terms “administering” or “administration” are intended to encompass all means for directly and indirectly delivering a compound to its intended site of action.

As used herein, the term “animal” means any animal, including mammals and, in particular, humans.

As used herein, the term “patient” refers to a mammal, particularly a human. Patients to be treated by the methods of the disclosed embodiments include both human subjects and animal subjects (e.g., dog, cat, monkey, chimpanzee, and/or the like) for veterinary purposes. The patients may be male or female and may be any suitable age, e.g., neonatal, infant, juvenile, adolescent, adult, or geriatric.

The terms “treat,” “treating” or “treatment,” and the like as used herein, refers to a method of alleviating or abrogating a disease and/or its attendant symptoms. For example, within the meaning of the present invention, the term “treat” also denotes to arrest, delay the onset (i.e., the period prior to clinical manifestation of a disease) and/or reduce the risk of developing or worsening a disease.

The terms “a” and “an,” when used to modify the ingredient of a composition, such as, active agent, buffering agent, and osmolyte, do not denote a limitation of quantity, but rather denote the presence of at least one of the referenced item. The term “or” or “and/or” is used as a function word to indicate that two words or expressions are to be taken together or individually. The terms “comprising,” “having,” “including,” and “containing” are to be construed as open-ended terms (i.e., meaning “including, but not limited to”). The endpoints of all ranges directed to the same component or property are inclusive and independently combinable.

Throughout the present specification, the terms “about” and/or “approximately” may be used in conjunction with numerical values and/or ranges. The term “about” is understood to mean those values near to a recited value. For example, “about 1200 [units]” may mean within ±10% of 1200, within ±10%, ±9%, ±8%, ±7%, ±7%, ±5%, ±4%, ±3%, ±2%, ±1%, less than ±1%, or any other value or range of values therein. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 70-70, etc.). Furthermore, all values within a given range may be an endpoint for the range encompassed thereby (e.g., the range 50-80 includes the ranges with endpoints such as 55-80, 50-75, etc.).

### 5.2 Pharmaceutical Formulations Comprising Nucleic Acids

One embodiment of the present invention is the development of low-viscosity, high concentration liquid formulations (HCLFs) of nucleic acids and their salts for convenient drug delivery to a patient. In particular, nucleic acid compositions which may be administered subcutaneously and/or which may require less frequent dosing than nucleic acid products currently on the market are investigated. In certain embodiments, high concentration nucleic acid formulations are self-administered on an out-patient basis. Some formulations of the invention have thixotropic and sheer thinning behaviors which are particularly preferred for subcutaneous and/or intramuscular administration. Formulations as provided herein offer improved tolerability, patient convenience during treatment and opportunity for outpatient dosing in comparison to currently available commercial nucleic acid formulations. In some embodiments, the viscosity of high concentration nucleic acid formulations provided herein decreases over time. In certain embodiments, the viscosity and/or fluidity of high concentration nucleic acid formulations provided herein decreases under an increase in shear strain. It should be understood that such properties are preferable for injectables and delivery devices, such as a syringe or preloaded subcutaneous device, in which the strain or shear stress the formulation is exposed to increases as the formulation passes from the barrel of the syringe/device through to the reduced orifice of the needle. In certain embodiments, the nucleic acid is defibrotide. Formulations of the invention, particularly those comprising defibrotide, may be used for the treatment of numerous conditions including, for example, treatment of peripheral arteriopathies, treatment of acute renal insufficiency, treatment of acute myocardial ischemia, treatment and prevention of Graft versus Host Disease (GvHD), treatment and prevention of Transplant-Associated Thrombotic Microangiopathy (TA-TMA), treatment of Ischemia Reperfusion Injury, such as for example, in solid organ transplantation (Kidney IRI for example), treatment and prevention of cytokine release syndrome (CRS) or Chimeric Antigen Receptor (CAR)-T Cell Related Encephalopathy Syndrome (CRES), and treatment and prevention of sinusoidal obstruction syndrome or VOD. In some embodiments, formulations of the invention, particularly those comprising defibrotide, may be administered to patients who have undergone, are undergoing, or are about to undergo, chemotherapy, stem cell ablation, and/or hematopoietic stem cell transplantation (HSCT). Other uses of defibrotide, methods for its production and testing are described in the following patents, patent applications and articles, each of which is hereby incorporated by reference in its entirety: U.S. Pat. Nos. 3,770,720; 3,829,567; 3,899,481; 4,693,134; 4,693,995; 4,938,873; 4,985,552; 5,081,109; 5,116,617; 5,223,609; 5,646,127; 5,646,268; 5,977,083; 6,046,172; 6,699,985; 6,767,554; 7,338,777; 8,551,967; 8,771,663, US Patent Publication Nos. 20080194506; 20090131362; 20110092576; 20130231470; 20140005256, U.S. patent application Ser. Nos. 14/019,674; 14/323,918; 14/408,272; 62/656,486; 62/657,161; 62/664,657; and International applications WO 2013/190582 and PCT/EP2015/077355. See also Palmer and Boa, Defibrotide. A Review of its pharmacodynamic and pharmacokinetic properties, and therapeutic use in vascular disorders, Drugs, 1993, February; 45(2):259-94; which is incorporated by reference herein. Other references cited throughout are also incorporated by reference in their entireties.

In certain embodiments, the defibrotide to be evaluated by the methods described herein are manufactured by a process such as that described in U.S. Pat. Nos. 4,985,552 and 5,223,609, both of which are hereby incorporated by reference in their entireties. In one preferred embodiment of the invention, defibrotide is a polydeoxyribonucleotide corresponding to the following formula of random sequence:

P1-5,(dAp)12-24,(dGp)10-20,(dTp)13-26,(dCp)10-20

[symbol]

wherein: P=phosphoric radical

dAp=deoxyadenylic monomer

dGp=deoxyguanylic monomer

dTp=deoxythymidylic monomer

dCp=deoxycytidylic monomer

The defibrotide as used herein may have one or more or all of the following chemico-physical properties: electrophoresis=homogeneous anodic mobility; extinction coefficient, E1 cm1% at 260±1 nm=220±10; extinction ratio, E230/E260=0.45±0.04; coefficient of molar extinction (referred to phosphorus), ε(P)=7.750±500; rotary power [α]D20°=53°±6; reversible hyperchromicity, indicated as % in native DNA, h=15±5; and a purine:pyrimidine ratio of 0.95±0.5.

One embodiment of the present invention comprises a nucleic acid formulation with various buffers or excipients, such as those found in Remington, The Science and Practice of Pharmacy (Remington the Science and Practice of Pharmacy) Twenty-Second Edition, 2013 Pharmaceutical Press which is hereby incorporated by reference in its entirety. See especially the monograph on Excipients starting at page 1837. Preferably, the nucleic acid is defibrotide. In some embodiments, a nucleic acid other than defibrotide is used.

In some embodiments, the invention includes a dipeptide buffer (e.g. L-Carnosine or glycylglycine). One preferred embodiment of the invention includes glycylglycine, which is a dipeptide of glycine. It is commercially available from supply houses, such as Sigma-Aldrich, and is useful as an excipient for biological systems. In specific embodiments of the present invention, glycylglycine is present at concentrations between about 1 mM to about 50 mM. In some embodiments, glycylglycine is present at concentrations between about 5 mM to about 100 mM, about 10 to about 60 mM, or about 10 to about 40 mM. In some embodiments, the glycylglycine is present at a concentration of about 1 mM, about 5 mM, about 10 mM, about 15 mM, about 20 mM, about 25 mM, about 30 mM, about 35 mM, about 40 mM, about 45 mM, or about 50 mM.

Other buffers or excipients can be present in the present formulation. In some embodiments, the low-viscosity pharmaceutical formulation comprises a buffer or excipient selected from sodium citrate, sodium succinate, histidine, TRIS buffer, HEPES buffer, sodium chloride, arginine, lidocaine, and/or polysorbate-80. In some embodiments, the low-viscosity formulation comprises a buffer or excipient so that the nucleic acid is in the form of an alkali metal salt. In some embodiments, the buffer or excipient includes a sodium salt. In some embodiments, the buffer or excipient is sodium citrate, sodium succinate, or sodium chloride.

In some embodiments, the buffer or excipient is sodium citrate, sodium succinate, or sodium chloride at a concentration of less than about 80 mM sodium salt. In some embodiments, the formulation comprises about 1-80 mM sodium salt. In some embodiments, the formulation comprises about 1 mM, about 5 mM, about 10 mM, about 15 mM, about 20 mM, about 25 mM, about 30 mM, about 35 mM, about 40 mM, about 45 mM, or about 50 mM sodium salt.

In some embodiments, the formulation comprises sodium citrate. In some embodiments, the sodium citrate is present at concentrations between about 5 to about 50 mM between about 5 to about 60 mM, about 10 to about 60 mM, or about 10 to about 40 mM. In some embodiments, the concentration of sodium citrate is about a 1 mM, about 5 mM, about 10 mM, about 15 mM, about 20 mM, about 25 mM, about 30 mM, about 35 mM, about 40 mM, about 45 mM, about 50 mM, about 55 mM, or about 60 mM.

Other excipients can be added to the present formulations, such as preservatives, salts, or pH adjusting agents.

In some embodiments of the invention, the viscosity of the low-viscosity formulation is between about 1 to about 70 cP. In some embodiments, the viscosity of the low-viscosity formulation is between about 5 cP to about 65 cP, or about 10 cP to about 65 cP. In some embodiments, the viscosity of the low-viscosity formulation is about 5 cP, about 10 cP, about 15 cP, about 20 cP, about 25 cP, about 30 cP, about 35 cP, about 40 cP, about 45 cP, about 50 cP, about 55 cP, about 60 cP, about 65 cP, or about 70 cP.

In some embodiments of the invention, viscosity of the low-viscosity formulation decreases over time. In some embodiments, the viscosity decreases during storage of the formulation. In some embodiments, the viscosity of the low-viscosity nucleic acid formulation provided herein decreases with decreasing mean molecular weight of the nucleic acid. In some embodiments, the viscosity of the low-viscosity nucleic acid formulation provided herein decreases with decreasing mean molecular weight of the nucleic acid at a given concentration of said nucleic acid. In some embodiments, the viscosity of the low-viscosity nucleic acid formulation provided herein decreases with decreasing mean molecular weight of the nucleic acid at a given concentration of said nucleic acid when viscosity is measured under room temperature conditions, such as from 15° C. to 35° C. In some embodiments, the viscosity decreases under increasing shear, agitation, and/or pressure. In some embodiments, the viscosity decreases during administration of the low-viscosity formulation (e.g. when passing through a needle). In some embodiments, the determination of the viscosity of the low-viscosity formulation varies depending on the temperature at which it is measured. In some embodiments, the viscosity of high concentration nucleic acid formulations provided herein decreases with decreasing mean molecular weight of the nucleic acid. In some embodiments, the viscosity of high concentration nucleic acid formulations provided herein increases with an increase in the mean molecular weight of the nucleic acid. In preferred embodiments, the viscosity is measured under room temperature conditions, such as from 15° C. to 35° C. More preferably, the viscosity is measured between 18° C. to 25° C. Even more preferably, the viscosity is measured at between 21° C. to 23° C.

In some embodiments, the low-viscosity formulations of the present disclosure have an osmolality between about 200 mOsm/kg and about 1000 mOsm/kg. In some embodiments, the low-viscosity formulations of the present disclosure have an osmolality between about 240 mOsm/kg to about 600 mOsm/kg or about 300 mOsm/kg to about 550 mOsm/kg. In some embodiments, the low-viscosity formulations of the present disclosure have an osmolality of about 200 mOsm/kg, about 240 mOsm/kg, about 250 mOsm/kg, about 300 mOsm/kg, about 350 mOsm/kg, about 400 mOsm/kg, about 450 mOsm/kg, about 500 mOsm/kg, about 600 mOsm/kg, about 650 mOsm/kg, about 700 mOsm/kg, about 750 mOsm/kg, about 800 mOsm/kg, about 850 mOsm/kg, about 900 mOsm/kg, or about 950 mOsm/kg.

In some embodiments, the present disclosure provides for methods for delivering the formulations of the disclosure. In certain embodiments, the formulations of the present invention are subcutaneously delivered. In some embodiments, formulations of the invention are administered subcutaneously by means of a device that can be used by the patient. In some embodiments, the low-viscosity formulation is a defibrotide formulation. In some embodiments, the formulation is a High Concentration Liquid Formulation (HCLF).

Devices for subcutaneous administration may be prefilled, with for example a predefined adult or pediatric dose, or may be used to administer a weight-based dose specific for individual patients. In some embodiments, the patient determines the dose and administers it. In certain embodiments, formulations of the invention are administered subcutaneously by means of a device that is commercially available such as, for example, the FREEDOM60® pump or similar (RMS™ Medical Products). In some specific embodiments, formulations of the invention are administered subcutaneously in less than about two hours, less than about one hour, or less than about 30 minutes. In some specific embodiments, formulations of the invention are delivered subcutaneously over about 5 minutes to about 1 hour, about 10 minutes to about 1 hour or about 15 minutes to about 45 minutes.

The formulation dosing may be determined by a variety of factors that will be readily apparent to a skilled artisan. In some embodiments, the dose is based on patient's baseline body weight. In some embodiments, formulation is administered in an amount of about 1 to about 100 mg per kilogram of body weight per day. For example the formulation is administered in an amount of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg per kilogram of body weight per day. In some embodiments, formulation is administered in an amount of about 25 mg per kilogram of body weight per day. In some embodiments, doses based on the patient's body weight are rounded to the nearest 10 mg for patients over 35 kg. In some embodiments, doses based on the patient's body weight were rounded to the nearest 5 mg for patients under 35 kg. In some embodiments, the formulation is a defibrotide formulation.

The formulation may be administered as a single daily dose or in multiple doses per day. In some embodiments, formulation is administered once a day. In some embodiments, formulation is administered in multiple doses per day. For example, the formulation may be administered in 2, 3, 4, 5, 6, 7, 8, 9, or in 10 doses per day. In some embodiments, the formulation is administered in four doses per day. In some embodiments, the formulation is administered in four doses per day every 6 hours.

In some embodiments, the dose and frequency of administration varies depending on route of administration. In some embodiments, subcutaneous administration of the low-viscosity formulations of the present disclosure allows for less-frequent administration and/or lower doses. In some embodiments, subcutaneous administration of the low-viscosity formulation of the present disclosure allows for reduced administration volume.

As a skilled artisan will appreciate, the treatment period may vary on a patient-by-patient basis. For example, in some embodiments, the treatment period is determined by monitoring signs and symptoms of hepatic VOD. For example, if the signs and symptoms of hepatic VOD are still present after an initial treatment period, defibrotide treatment is continued until resolution of VOD. In some embodiments, if the signs and symptoms of hepatic VOD are still present after 21 days, defibrotide treatment is continued until resolution of VOD up to a maximum of 60 days. Thus, in certain embodiments, the treatment period may last anywhere from 21 to 60 days. For example, the treatment period lasts for 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the treatment period lasts 21 days.

In some embodiments, administration of the formulations of the present disclosure treats or ameliorates development of VOD and/or VOD symptoms compared to an untreated patient or the same patient before formulation administration. In some embodiments, VOD and/or VOD symptoms are treated or ameliorated in the patient between day 1 and year 10. In some embodiments, administration of the formulation treats or ameliorates development of VOD and/or VOD symptoms compared to an untreated patient or the same patient before defibrotide administration at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the formulation treats or ameliorates development of VOD and/or VOD symptoms compared to an untreated patient or the same patient before formulation administration for about 1 day, about 1 week, about 1 month, about 2 months, about 3 months, about 4 months, about 5 months, about 6 months, about 1 year, about 2 years, about 5 years, or about 10 years, or more.

In some embodiments, administration of the formulations of the present disclosure treats or ameliorates VOD and/or VOD symptoms by about 1%, about 5%, about 10%, about 20%, about 30%, about 40%, about 50%, about 60%, about 70%, about 80%, about 90%, or about 100% compared to an untreated patient or the same patient before formulation administration. In some embodiments, administration of the formulation treats or ameliorates development of VOD and/or VOD symptoms compared to an untreated patient or the same patient before formulation administration by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the formulation treats or ameliorates development of VOD and/or VOD symptoms compared to an untreated patient or the same patient before formulation administration by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the and scope of the invention.

### 6. EXAMPLES

### 6.1 Example 1—Identification of Limiting Solution Attributes

In order to develop High Concentration Liquid Formulations (HCLFs) of a nucleic acid, it is important to identify key physicochemical properties of the solution that may limit formulation. To investigate this, a number of solutions ranging in defibrotide concentrations of up to approximately 300 mg/mL were generated using two different formulations and the solution properties of each were characterized as a function of defibrotide concentration. The visual appearance, solubility, viscosity, osmolality, polymer structure in solution (far- and near-UV Circular Dichroism), thermal properties (Differential Scanning Calorimetry), and molecular weight/aggregation (DLS, FTIR, and SEC-MALS) were some of the properties analyzed for the various defibrotide solutions.

Sample Preparation: defibrotide was formulated in 34 mM sodium citrate, pH 7.3 or 34 mM glycylglycine (“Gly-Gly”), pH 7.5 by centrifugal concentration or by dissolving defibrotide API at 80, 150, 200, 250, and 300 mg/mL. Product concentration was typically measured by Ultraviolet-Visible Spectroscopy. Defibrotide samples were diluted gravimetrically in triplicate to a target concentration of 0.1 mg/mL in their respective formulation buffers using 35 μL of sample. A₂₆₀ and A₃₂₀ were measured using a cuvette path length of 0.2 cm. A₂₆₀ values were corrected for light scattering at 320 nm and the concentration was determined using an extinction coefficient of 22.2 mL*mg⁻¹ cm⁻¹. A sample density of 1.08 g/mL and a diluent density of 1.0 g/mL were used to correct the mass of the sample when determining the dilution factor. The physiochemical properties of each solution were analyzed as a function of concentration by the following methods:

Visual Appearance & Turbidity: digital color matching was performed by a Core Module 3 (“CM3”) robot. The color of defibrotide samples was also evaluated using the European Pharmacopeia (EP) color matching analysis using seven EP color standards, BY1-BY7, with BY1 being the most intensely colored standard. The analysis was conducted under a light box with a white background (Eisai Machinery Observation Lamp Model MIH-DX, Fisher light Meter Model 06-662-63). The color evaluations for the Gly-Gly and citrate formulations were not significantly different; all were clear slightly yellow, or brown-yellow solutions with the intensity of coloration being more pronounced than the standard. In addition, no visible particles were detected (particle sizes of ≥80 μm were evaluated). Turbidity was also measured against seven turbidity standards. The color of all formulations when compared to the EP color standards was BY4 at the initial time point as well as after one month of storage at 25±2° C./60±5% RH showing that all formulations were stable for up to at least three months at 25±2° C./60±5% RH.

Solubility: the solubility of defibrotide in solution was evaluated via polyethylene glycol (PEG) precipitation using the CM3 robot for analysis. Throughout the studies, a miniscule amount of precipitation was observed even in the presence of a high quantity PEG, thus indicating high solution solubility of the product.

Solution Viscosity: the solution viscosity of defibrotide samples were analyzed at approximately 80, 150, 200, 250, and 300 mg/mL concentrations. Typically a Brookfield DV-III Ultra Programmable Rheometer was used to measure the viscosity. The samples were analyzed neat at 22° C. using approximately 550 μL. The viscosity of Defitelio® was 3.9 cP with no dependence on shear rate. The results suggested that Defitelio® displayed Newtonian fluid behavior. Defibrotide formulations of the invention formulated at 300 mg/mL in citrate and Gly-Gly buffers demonstrated that the viscosity was dependent of shear rate and product concentration. The viscosity of the formulated defibrotide appeared to increase exponentially as a function of the product concentration. The viscosity of defibrotide in 34 mM Gly-Gly, pH 7.5 as a function of product concentration was significantly lower compared to defibrotide in 34 mM sodium citrate and pH 7.3 demonstrating the ability of Gly-Gly to improve the solution properties of defibrotide in the HCLFs of the invention.

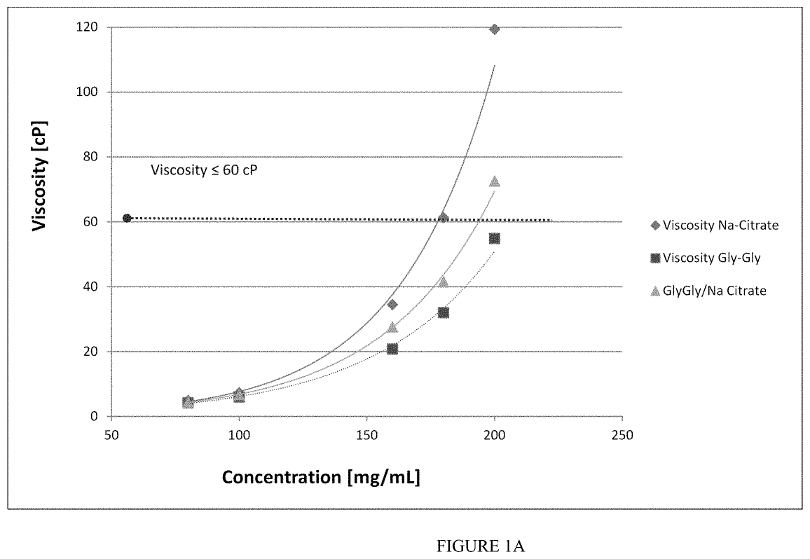

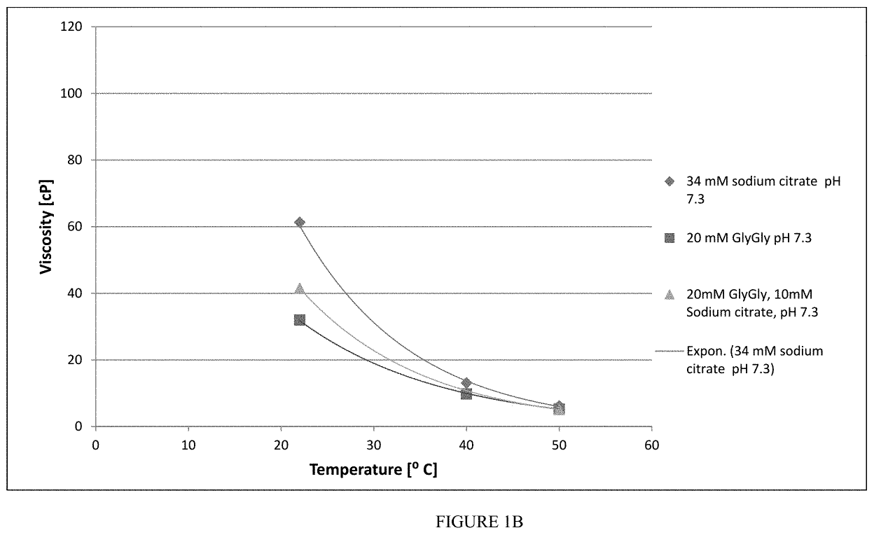

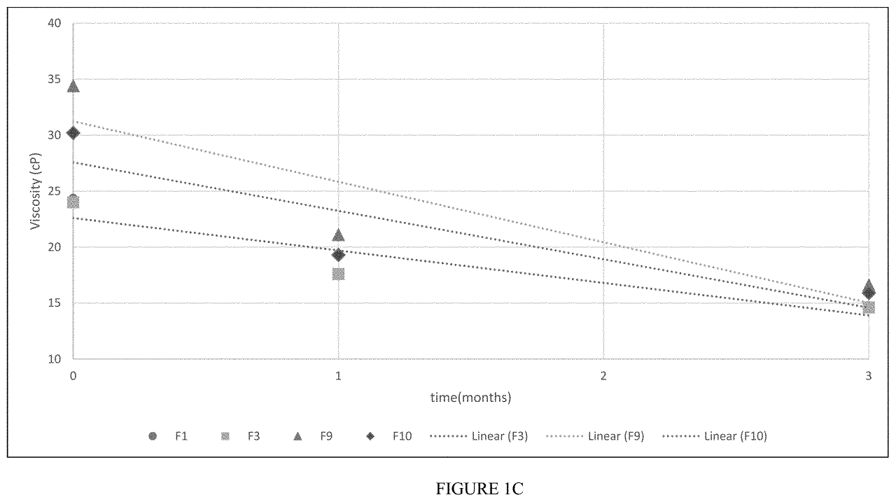

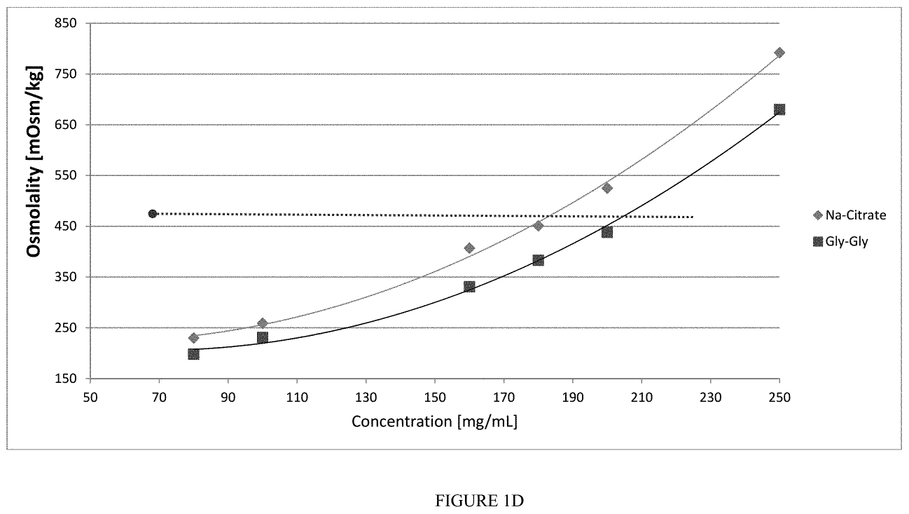

Osmolality: The osmolality was measured by at least two different methods and results are reported in the Figures throughout (see, for example, FIG. 1D). Typically, a Vapro Vapor Pressure Osmometer was used for one measurement. The osmolality of defibrotide in 34 mM Gly-Gly, pH 7.5 was lower compared to defibrotide in 34 mM sodium citrate and pH 7.3 demonstrating the ability of Gly-Gly to improve the solution properties of defibrotide in the HCLFs of the invention.

Far and Near-Ultraviolet Circular Dichroism: the secondary and tertiary structure of defibrotide formulations in solution, as a function of product concentration, was assessed by circular dichroism and analyzed on a Jasco J-810 Spectropolarimeter.

Free Nucleic Bases Analysis: Samples were quantitatively prepared at 1.6 mg/mL with mobile phase (50 mM CH₃COONH4, pH 5.0) and analyzed by RP-HPLC using a detection wavelength of 254 nm. A Synergi Fusion 4 μm-RP 80 Å column was used to separate the nucleic base using a flow rate of 1 mL/min. Defitelio® was used as a reference and was prepared at 1.6 mg/mL in mobile phase.

Fourier Transform Infrared Spectroscopy: FTIR analysis was performed using standard techniques to evaluate the structure of HCLFs of defibrotide. The FTIR analysis demonstrated that the two defibrotide formulations (citrate and Gly-Gly) at 300 mg/mL displayed a similar profile when compared to Defitelio®.

Differential Scanning Calorimetry: the thermal properties in solution of defibrotide were measured by differential scanning calorimetry using standard techniques. The results suggest that concentration and/or buffer matrix, including Gly-Gly, can influence the thermal properties of defibrotide.

Size Distribution: measured for each formulation as a function of the product concentration in order to account for the molecular weight of contributing structures to the molecular weighted average. The polydispersity index (Mw/Mn) was used to measure the heterogeneity of the formulations and, based on the results, the samples were concluded to be polydispersed. The results showed that defibrotide formulated at 300 mg/mL in citrate and Gly-Gly is comparable to Defitelio®.

Overall, the results using the above methods indicate that the solution osmolality and viscosity are important formulation attributes playing a critical role in limiting how high product concentration can be achieved that is well tolerated. These attributes in Gly-Gly containing formulations demonstrated notable solution properties improvements which also correlate with thermal attributes in solution (ΔH, Tm).

The graph in FIG. 1A shows the viscosity of formulations made using increasing defibrotide concentrations in the presence of sodium citrate, Gly-Gly or a mixture of the two. The results show that the viscosity of defibrotide formulations is strongly dependent on its concentration, and a 200 mg/mL solution has roughly 10-fold higher viscosity as compared to the 80 mg/mL solution.

The graph is FIG. 1B shows the viscosity as a function of temperature in three different formulations comprising either sodium citrate, Gly-Gly or a mixture of the two.

The graph in FIG. 1C shows the viscosity decrease over the course of time in these selected formulations: 20 mM GlyGly (blue circles; overlapped by the orange squares), 20 mM GlyGly and 34 mM sodium citrate (orange squares), 20 mM GlyGly and 100 mM sodium succinate (blue triangles) and 20 mM GlyGly and 20 mM sodium chloride (red diamonds). GlyGly containing formulations show the lowest viscosity for a given time point.

The graph in FIG. 1D shows the osmolality of formulations made using increasing defibrotide concentrations in the presence of Gly-Gly or sodium citrate buffers.

### 6.2 Example 2—Effect of Buffers on Formulation Properties

### 6.2.1 Example 2.1—Effect of Buffers and Excipients on Viscosity & Osmolality

Increasing the defibrotide concentration was shown in Example 1 to increase both viscosity and osmolality. It is important for pharmaceutical preparations for parenteral administration to be of low-viscosity and/or isotonic. In order to identify buffers or excipients that may lower the viscosity and/or osmolality of defibrotide formulations, a wide-panel screening of various buffers and excipients (including GRAS excipients) was performed using a 200 mg/mL defibrotide formulation.

Test formulations were prepared to target 200 mg/mL as shown in Table 1 below.

TABLE 1
Defibrotide Formulations using Various Buffers and Excipients

| | Defibrotide | | | | |
|---|---|---|---|---|---|
| | Concen- | | Average | | Shear |
| | tration | Osmolality | Viscosity | Viscosity | Rate |
| Formulation | (mg/mL) | (mmol/kg) | (cP) | (cP) | (s⁻¹) |
| Defitelio DP, 34 | 80 | Not Tested | 4.2 | 4.21 | 113 |
| mM Sodium | | | | 4.24 | 225 |
| Citrate, pH 7.3 | | | | 4.23 | 338 |
| | | | | 4.28 | 525 |
| 34 mM Sodium | 200 | 438 | 29.6 | 29.6 | 15 |
| Citrate, pH 7.3 | | | | 29.4 | 30 |
| | | | | 29.8 | 48.8 |
| | | | | 29.7 | 71.3 |
| 34 mM Sodium | | 544 | 58 | 61.9 | 7.5 |
| Citrate, pH 6.5 | | | | 57.9 | 22.5 |
| | | | | 56.5 | 30 |
| | | | | 55.5 | 37.5 |
| 34 mM Sodium | | 636 | 51.9 | 51.8 | 7.5 |
| Citrate, 100 mM | | | | 51.8 | 22.5 |
| NaCl, pH 7.3 | | | | 52 | 30 |
| | | | | 51.9 | 37.5 |
| 34 mM Sodium | | 643 | 46.1 | 46.6 | 3.45 |
| Citrate, 100 mM | | | | 45.5 | 15 |
| Arginine, pH 7.3 | | | | 46 | 30 |
| | | | | 46.2 | 45 |
| 34 mM Sodium | | 542 | 53.6 | 52.9 | 11.3 |
| Citrate, 0.1% | | | | 53.7 | 22.5 |
| PS-80, pH 7.3 | | | | 53.9 | 33.8 |
| | | | | 53.8 | 38.3 |
| 34 mM Sodium | | 994 | 38.3 | 38.3 | 15 |
| Citrate, 250 mM | | | | 38.2 | 30 |
| Lidocaine HCl, | | | | 38.4 | 37.5 |
| pH 7.0 | | | | 38.4 | 52.5 |
| 34 mM Sodium | | 435 | 46.4 | 46.1 | 15 |
| Citrate, pH 8.0 | | | | 46.3 | 22.4 |
| | | | | 46.4 | 30 |
| | | | | 46.6 | 45 |
| 34 mM Gly-Gly, | | 566 | 38.5 | 38.6 | 15 |
| 100 mM NaCl, | | | | 38.4 | 30 |
| pH 7.5 | | | | 38.5 | 37.5 |
| | | | | 38.6 | 54 |
| 34 mM Gly-Gly, | 200 | 560 | 39.7 | 39.4 | 15 |
| 100 mM | | | | 39.5 | 30 |
| Arginine, pH 7.5 | | | | 39.7 | 45 |
| | | | | 40.3 | 52.5 |
| 34 mM Gly-Gly, | | 359 | 38.2 | 37.9 | 15 |
| 0.1% PS-80, | | | | 38.2 | 30 |
| pH 7.5 | | | | 38.2 | 45 |
| | | | | 38.4 | 54 |
| 34 mM Gly-Gly, | | 950 | 34.1 | 34 | 15 |
| 250 mM | | | | 34.1 | 37.5 |
| Lidocaine HCl, | | | | 34.2 | 48.8 |
| pH 7.0 | | | | 34.6 | 60 |
| 34 mM Gly-Gly, | | 323 | 27.7 | 27.9 | 16.5 |
| pH 7.5 | | | | 27.7 | 31.5 |
| | | | | 27.6 | 51 |
| | | | | 27.7 | 75 |
| 34 mM Gly-Gly, | | 370 | 36.9 | 36.5 | 15 |
| pH 8.0 | | | | 37 | 33.8 |
| | | | | 37 | 45 |
| | | | | 37.2 | 56.3 |
| 34 mM Gly-Gly, | | 375 | 34 | 33.7 | 15 |
| pH 8.5 | | | | 33.7 | 37.5 |
| | | | | 34.1 | 48.8 |
| | | | | 34.3 | 60.8 |
| 34 mM Tris, pH | | 394 | 39.4 | 39.1 | 15 |
| 7.5 | | | | 39.2 | 30 |
| | | | | 39.4 | 45 |
| | | | | 39.8 | 52.5 |
| 34 mM HEPES, | | 379 | 43.9 | 43.8 | 15 |
| pH 7.5 | | | | 43.8 | 30 |
| | | | | 43.7 | 37.5 |
| | | | | 44.2 | 46.5 |
| 34 mM His, pH | | 364 | 37 | 36.9 | 15 |
| 7.3 | | | | 36.9 | 30 |
| | | | | 37 | 45 |
| | | | | 37.3 | 56.3 |

TABLE 2
Solution viscosity and osmolality of defibrotide in Gly-Gly containing buffer in
comparison to sodium citrate as a function of product concentration

| | | | | Mean | Shear | |
|---|---|---|---|---|---|---|
| | Concentration | Temperature | Viscosity | Viscosity | Rate | Osmolality |
| Formulation | (mg/mL) | (° C.) | (cP) | (cP) | (s⁻¹) | Mmol/kg |
| 34 mM | 80 | 2-8° C. | 11.5 | 11.6 | 37.5 | |
| Sodium | | | 11.6 | | 75.0 | |
| Citrate pH | | | 11.6 | | 113 | |
| 7.3 | | | 11.8 | | 150 | |
| | | 22° C. | 4.9 | 4.9 | 60.0 | 230 |
| | | | 4.9 | | 150.0 | |
| | | | 4.9 | | 263.0 | |
| | | | 4.9 | | 413.0 | |
| | | 40° C. | 2.3 | 2.3 | 113.0 | |
| | | | 2.3 | | 300.0 | |
| | | | 2.3 | | 450.0 | |
| | | | 2.4 | | 900.0 | |
| | | 50° C. | 1.56 | 1.6 | 225 | |
| | | | 1.58 | | 450 | |
| | | | 1.58 | | 900 | |
| | | | 1.60 | | 1200 | |
| | 100 | 2-8° C. | 20.2 | 20.7 | 22.5 | |
| | | | 20.5 | | 37.5 | |
| | | | 20.9 | | 75.0 | |
| | | | 21.2 | | 97.5 | |
| | | 22° C. | 7.5 | 7.3 | 37.5 | 259 |
| | | | 7.2 | | 75.0 | |
| | | | 7.2 | | 150 | |
| | | | 7.2 | | 300 | |
| | | 40° C. | 3.0 | 3.0 | 113 | |
| | | | 3.0 | | 263 | |
| | | | 3.0 | | 413 | |
| | | | 3.1 | | 675 | |
| | | 50° C. | 1.96 | 2.0 | 150 | |
| | | | 1.95 | | 300 | |
| | | | 1.98 | | 675 | |
| | | | 1.99 | | 975 | |
| | 160 | 2-8° C. | 628.3 | 655.5 | 0.750 | |
| | | | 648.1 | | 1.50 | |
| | | | 669.9 | | 2.25 | |
| | | | 675.5 | | 3.00 | |
| | | 22° C. | 34.6 | 34.5 | 15.0 | 407 |
| | | | 34.4 | | 22.5 | |
| | | | 34.4 | | 37.5 | |
| | | | 34.4 | | 60.0 | |
| | | 40° C. | 8.2 | 8.6 | 37.5 | |
| | | | 8.3 | | 75.0 | |
| | | | 8.4 | | 150 | |
| | | | 9.4 | | 225 | |
| | | 50° C. | 4.26 | 4.4 | 75.0 | |
| | | | 4.31 | | 156 | |
| | | | 4.39 | | 300 | |
| | | | 4.50 | | 450 | |
| | 180 | 2-8° C. | N/A | <600 | N/A | |
| | | | N/A | | N/A | |
| | | | N/A | | N/A | |
| | | | N/A | | N/A | |
| | | 22° C. | 61.8 | 61.3 | 4.50 | 451 |
| | | | 60.7 | | 7.50 | |
| | | | 61.4 | | 15.0 | |
| | | | 61.3 | | 30.0 | |
| | | 40° C. | 11.5 | 13.1 | 37.5 | |
| | | | 12.6 | | 75.0 | |
| | | | 13.5 | | 113.0 | |
| | | | 14.8 | | 150.0 | |
| | | 50° C. | 5.85 | 6.2 | 75.0 | |
| | | | 6.01 | | 150 | |
| | | | 6.31 | | 225 | |
| | | | 6.47 | | 300 | |
| | 200 | 2-8° C. | N/A | <600 | N/A | |
| | | | N/A | | N/A | |
| | | | N/A | | N/A | |
| | | | N/A | | N/A | |
| | | 22° C. | 117.1 | 119.4 | 3.75 | 525 |
| | | | 119.2 | | 6.00 | |
| | | | 119.9 | | 11.3 | |
| | | | 121.5 | | 15.0 | |
| | | 40° C. | 17.3 | 18.3 | 22.5 | |
| | | | 17.9 | | 52.5 | |
| | | | 18.4 | | 75.0 | |
| | | | 19.5 | | 113.0 | |
| | | 50° C. | 7.26 | 7.5 | 75.0 | |
| | | | 7.34 | | 150 | |
| | | | 7.42 | | 225 | |
| | | | 7.88 | | 263 | |
| | 250 | 2-8° C. | N/A | <600 | N/A | |
| | | | N/A | | N/A | |
| | | | N/A | | N/A | |
| | | | N/A | | N/A | |
| | | 22° C. | N/A | <600 | N/A | 792 |
| | | | N/A | | N/A | |
| | | | N/A | | N/A | |
| | | | N/A | | N/A | |
| | | 40° C. | N/A | <600 | N/A | |
| | | | N/A | | N/A | |
| | | | N/A | | N/A | |
| | | | N/A | | N/A | |
| | | 50° C. | 31.60 | 28.5 | 7.50 | |
| | | | 27.60 | | 37.5 | |
| | | | 27.20 | | 52.5 | |
| | | | 27.70 | | 75.0 | |
| 20 mM Gly- | 80 | 2-8° C. | 8.95 | 9.1 | 37.5 | |
| Gly pH 7.3 | | | 9.10 | | 75.0 | |
| | | | 9.15 | | 150 | |
| | | | 9.18 | | 188 | |
| | | 22° C. | 4.1 | 4.2 | 75.0 | 198 |
| | | | 4.1 | | 150.0 | |
| | | | 4.2 | | 225.0 | |
| | | | 4.2 | | 413.0 | |
| | | 40° C. | 2.3 | 2.4 | 113.0 | |
| | | | 2.3 | | 300.0 | |
| | | | 2.4 | | 450.0 | |
| | | | 2.4 | | 825.0 | |
| | | 50° C. | 1.61 | 1.6 | 225 | |
| | | | 1.61 | | 450 | |
| | | | 1.62 | | 900 | |
| | | | 1.64 | | 1200 | |
| | 100 | 2-8° C. | 14.2 | 14.3 | 37.5 | |
| | | | 14.3 | | 75.0 | |
| | | | 14.4 | | 113.0 | |
| | | | 14.4 | | 150.0 | |
| | | 22° C. | 6.0 | 6.0 | 75.0 | 231 |
| | | | 6.0 | | 113.0 | |
| | | | 6.0 | | 188.0 | |
| | | | 6.0 | | 300.0 | |
| | | 40° C. | 2.9 | 2.9 | 113.0 | |
| | | | 2.9 | | 263.0 | |
| | | | 2.9 | | 413.0 | |
| | | | 2.9 | | 675.0 | |
| | | 50° C. | 2.02 | 2.1 | 156 | |
| | | | 2.05 | | 300 | |
| | | | 2.08 | | 675 | |
| | | | 2.11 | | 900 | |
| | 160 | 2-8° C. | 88.1 | 88.0 | 6.00 | |
| | | | 86.7 | | 7.50 | |
| | | | 88.5 | | 11.3 | |
| | | | 88.6 | | 22.5 | |
| | | 22° C. | 20.8 | 20.8 | 15.0 | 331 |
| | | | 20.7 | | 37.5 | |
| | | | 20.8 | | 75.0 | |
| | | | 20.8 | | 97.5 | |
| | | 40° C. | 7.2 | 7.3 | 37.5 | |
| | | | 7.3 | | 75.0 | |
| | | | 7.3 | | 188.0 | |
| | | | 7.5 | | 263 | |
| | | 50° C. | 4.10 | 4.2 | 150 | |
| | | | 4.09 | | 300 | |
| | | | 4.18 | | 450 | |
| | | | 4.27 | | 488 | |
| | 180 | 2-8° C. | N/A | <600 | N/A | |
| | | | N/A | | N/A | |
| | | | N/A | | N/A | |
| | | | N/A | | N/A | |
| | | 22° C. | 31.6 | 32.0 | 7.50 | 383 |
| | | | 32.2 | | 15.0 | |
| | | | 32.0 | | 37.5 | |
| | | | 32.2 | | 60.0 | |
| | | 40° C. | 9.6 | 9.8 | 37.5 | |
| | | | 9.7 | | 75.0 | |
| | | | 9.9 | | 113.0 | |
| | | | 10.2 | | 188.0 | |
| | | 50° C. | 5.24 | 5.3 | 75.0 | |
| | | | 5.26 | | 150 | |
| | | | 5.33 | | 300 | |
| | | | 5.43 | | 375 | |
| | 200 | 2-8° C. | N/A | <600 | N/A | |
| | | | N/A | | N/A | |
| | | | N/A | | N/A | |
| | | | N/A | | N/A | |
| | | 22° C. | 54.0 | 54.9 | 7.50 | 438 |
| | | | 55.4 | | 11.3 | |
| | | | 55.0 | | 18.8 | |
| | | | 55.0 | | 33.8 | |
| | | 40° C. | 14.0 | 14.2 | 22.5 | |
| | | | 14.2 | | 52.5 | |
| | | | 14.2 | | 90.0 | |
| | | | 14.3 | | 127.0 | |
| | | 50° C. | 7.17 | 7.4 | 75.0 | |
| | | | 7.31 | | 150 | |
| | | | 7.43 | | 22.5 | |
| | | | 7.56 | | 263 | |
| | 250 | 2-8° C. | N/A | <600 | N/A | |
| | | | N/A | | N/A | |
| | | | N/A | | N/A | |
| | | | N/A | | N/A | |
| | | 22° C. | N/A | <600 | N/A | 680 |
| | | | N/A | | N/A | |
| | | | N/A | | N/A | |
| | | | N/A | | N/A | |
| | | 40° C. | 46.00 | 44.5 | 15.0 | |
| | | | 44.40 | | 30.0 | |
| | | | 44.00 | | 37.5 | |
| | | | 43.50 | | 41.3 | |
| | | 50° C. | 23.00 | 22.6 | 37.5 | |
| | | | 22.00 | | 52.5 | |
| | | | 22.40 | | 75.0 | |
| | | | 22.80 | | 90.0 | |

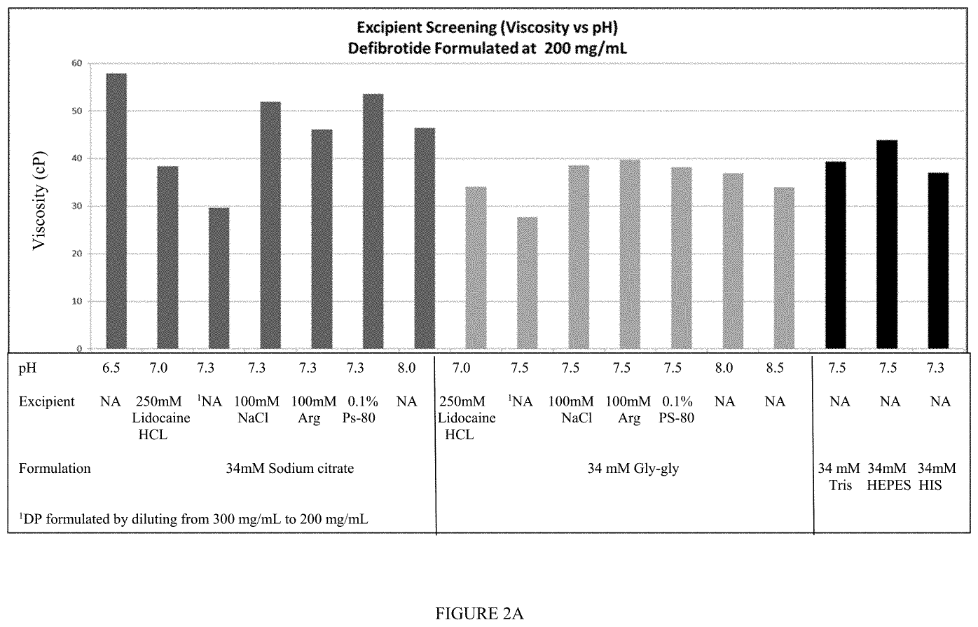

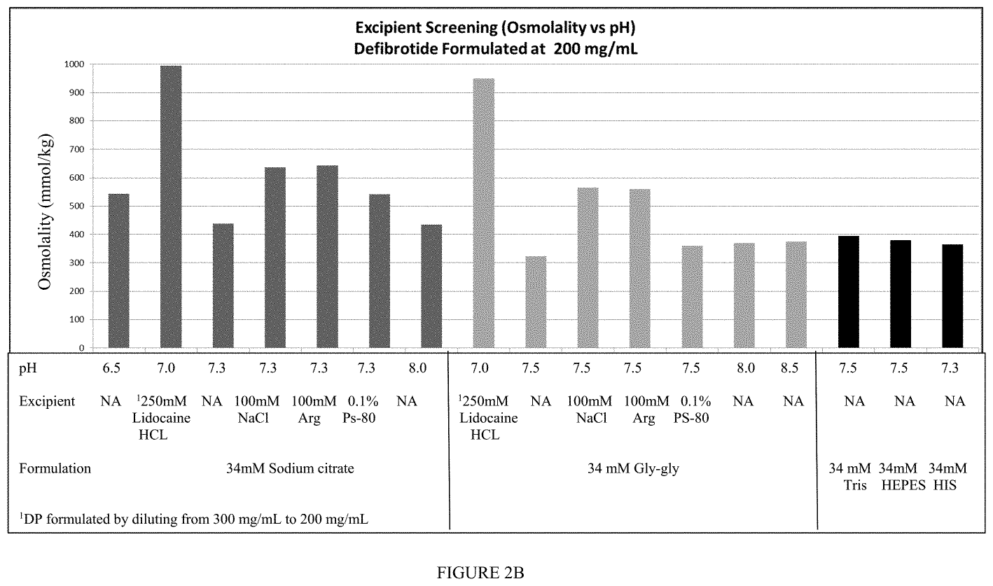

As shown in Tables 1 and 2 as well as the graphs in FIGS. 1B, 1C, and 2A, the HCLF formulations containing Gly-Gly had significantly lower viscosity overall compared to other formulations and when tested under different pH, product concentration, and temperature conditions (see FIG. 2A) as compared to the citrate buffer. Notably, the TRIS and histidine buffers also had lower viscosity (less than 40 cP) at 200 mg/mL defibrotide. For nearly all of the formulations in Gly-Gly, the viscosity was up to 50% lower compared to the citrate buffer for given product concentration and ambient temperature conditions. Ambient temperatures may change depending on the region and therefore, viscosity is preferably measured between about 15° C. to 30° C.; however, it may be slightly higher or lower given different weather conditions. For example, one preferred formulation containing 180 mg/mL of defibrotide (having a mean molecular weight of 14 kDA), and containing 20 mM Gly-Gly and 34 mM sodium citrate pH 7.0, had a viscosity of 12 cP when measured at 25° C. Other formulations had a viscosity of 27 cp when defibrotide having a mean molecular weight of 17 kDa was used under the same conditions. Out of the excipients screened, only lidocaine showed a potential for further reduction of viscosity; however, it increased osmolality >900 mOsm/kg (see FIG. 2B) and therefore was not considered practical for further investigation. Gly-gly buffer showing the lowest viscosity overall was identified as a preferred buffer for 200 mg/mL HCLF defibrotide formulations.

### 6.2.2 Example 2.2—Effect of Different Sodium Ion Sources on Buffering

### Capacity and Stability During Storage

In order to compare the buffering capacity of various buffer solutions in high concentration defibrotide (“DF”) formulations containing the Gly-Gly buffer system, sixteen different buffers, utilizing three different sodium ion sources were used to evaluate the stability, impurity profile, and solution properties of the DF formulations. A summary of these formulations is shown in Table 3.

TABLE 3
Summary of Defibrotide Formulations

| | | | Sodium | Sodium | |
|---|---|---|---|---|---|
| Formulation | Key-753 | Gly-Gly | Citrate | Succinate | NaCl |
| Code | (mg/mL) | (mM) | (mM) | (mM) | (mM) |
| F1 | 180 | 20 | — | — | — |
| F2 | 180 | 20 | 20 | — | — |
| F3 | 180 | 20 | 34 | — | — |
| F4 | 180 | 20 | 80 | — | — |
| F5 | 180 | 20 | 100 | — | — |
| F6 | 180 | 20 | — | 20 | — |
| F7 | 180 | 20 | — | 34 | — |
| F8 | 180 | 20 | — | 80 | — |
| F9 | 180 | 20 | — | 100 | — |
| F10 | 180 | 20 | — | — | 20 |
| F11 | 180 | 20 | — | — | 34 |
| F12 | 180 | 20 | — | — | 80 |
| F13 | 180 | 20 | — | — | 100 |
| F14 | 180 | 20 | 40 | — | 40 |
| F15 | 160 | 20 | 40 | — | 40 |
| F16 | 140 | 20 | 40 | — | 40 |

Based on the appearance, color, and clarity results, defibrotide formulated in the Gly-Gly buffers were stable following storage at 25±2° C./60±5% RH for up to at last three months.

UV and pH analysis showed that the defibrotide concentration remained constant for all formulations when stored at 25±2° C./60±5% RH for up to three months.

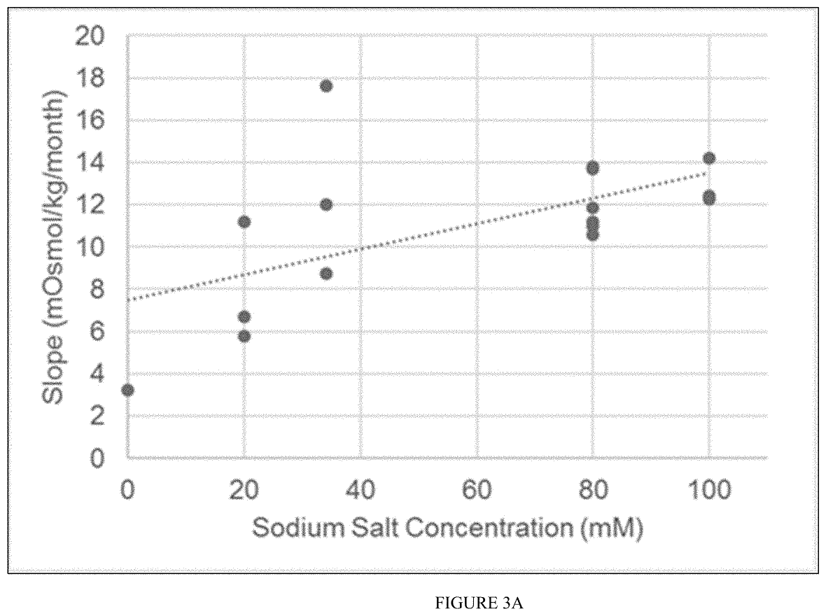

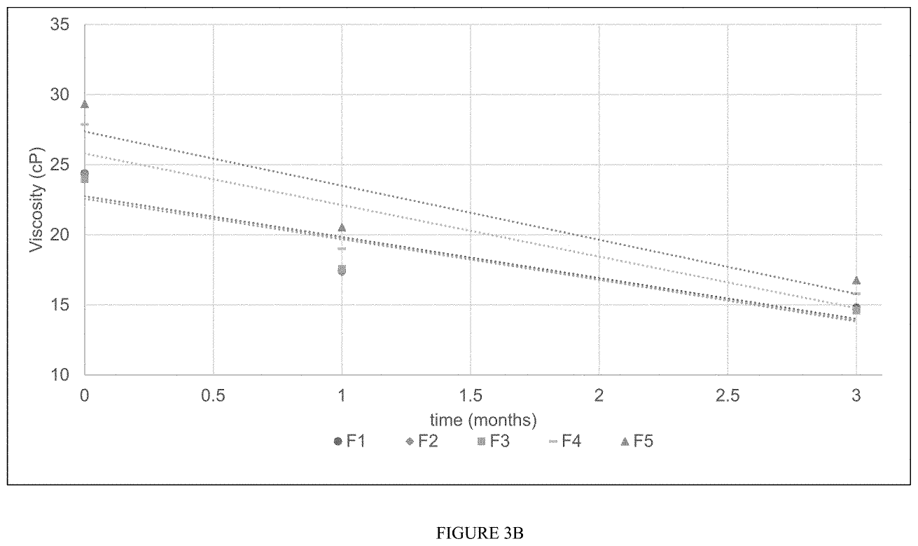

The viscosity of all formulations decreased as a function of time (see for example, FIG. 3B). The viscosity at the initial time point were within the range of 23.8 cP-34.4 cP for all formulations at 180 mg/mL and decreased by up to approximately 52% after storage at 25±2° C./60±5% RH for three months. The formulations F1-F5 as listed in FIG. 3B are described in Table 3 above.

A small osmolality increase trend was observed as a function of storage time that correlated with sodium salt concentration. As salt concentration was increased, the change in increase in osmolality was greater (see FIG. 3A). Formulations with less than 80 mM total sodium salt had the lowest change in osmolality over time and were under 500 mOsm/kg.

The stability indicating FNB assay demonstrated that total impurities and free nucleic bases increased slightly after storage for one month. Overall, formulations F2 and F3 had the lowest amount of free nucleic bases.

Size Exclusion Chromatograph (SEC)-Multi-Angle Light Scattering (MALS) analysis was performed to determine the size distribution and molecular weight of defibrotide as a function of the product concentration. DF formulations and API reference material were diluted to 4 mg/mL in SEC mobile phase in a glass screw cap tube (10 mL). The solution was maintained at room temperature for one hour without stirring. Subsequently, the sample solution was heated to approximately 100° C. (boiling water) and maintained at this temperature for 15 minutes. Finally, the sample solution was cooled using water and ice for five minutes. After stabilization at room temperature (about 15 minutes), the samples were filtered with a 0.20 μm SFCA syringe filter. The sample solution was analyzed by SEC-MALS within one hour of preparation. Reference material was prepared from defibrotide API at 4 mg/mL in mobile phase. The analysis indicated that all formulations have similar sizes and polydispersity

Based on these combined results 180 mg/mL defibrotide in 20 mM Gly-Gly and less than 80 mM sodium salt (and preferably 20-34 mM sodium citrate) are preferred buffer combinations for HCLF formulations.

### 6.3 Example 3—Viscosity Change Over Time and as a Function of Temperature

It is important for pharmaceutical products to maintain their integrity over time to allow for a suitable shelf-life. The viscosity of 200 mg/mL defibrotide formulations using Gly-Gly buffer were therefore measured as a function of both time and temperature.

Samples were prepared as described above.

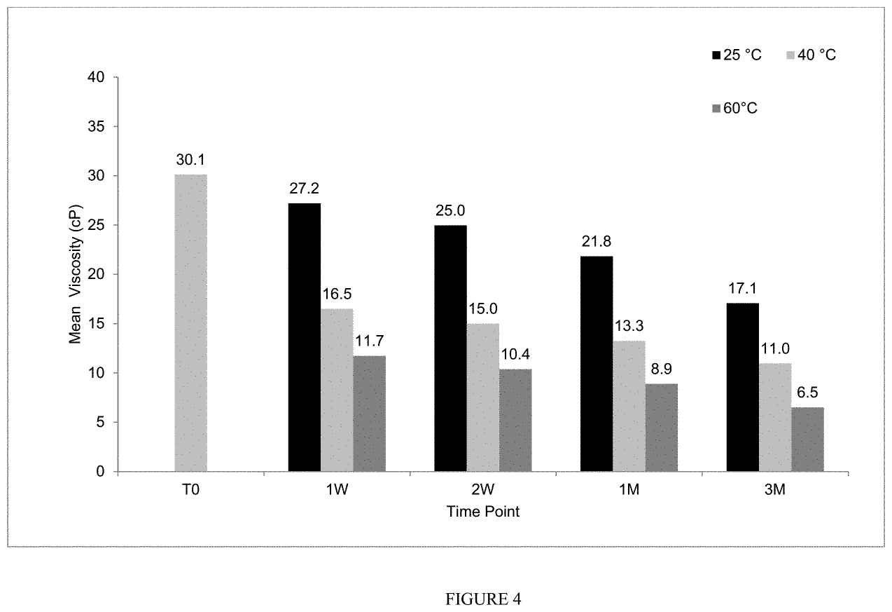

The graph in FIG. 4 shows that the viscosity of 200 mg/mL defibrotide formulations using 20 mM Gly-Gly buffer decreases over time and also decreases with increasing temperature (measured at 25° C., 40° C. and 60° C.). The viscosity decrease as a function of temperature and over time is favorable for drug delivery and product manufacturing, particularly for high concentration products such as HCLFs. The decrease of the viscosity over time, thixotropic behavior, is especially favorable and leads to improved patient convenience and tolerability of these formulations. Defibrotide is a temperature stable product thus the decrease of viscosity at higher temperatures provides additional opportunities for improved patient convenience. For example, if a patient warms up the formulation prior to administration, the viscosity will go down allowing for continued ease of administration particularly for subcutaneous and/or intramuscular administration.

### 6.4 Example 4—Osmolality Over Time Using Forced Degradation

It is also important for pharmaceutical products to maintain low osmolality over time and under various conditions. The osmolality of 200 mg/mL defibrotide formulations using Gly-Gly and citrate buffers were therefore measured as a function of both time and temperature using forced degradation studies.

Samples were prepared as described above. Osmolality of the formulations was measured at 25° C., 40° C. and 60° C.

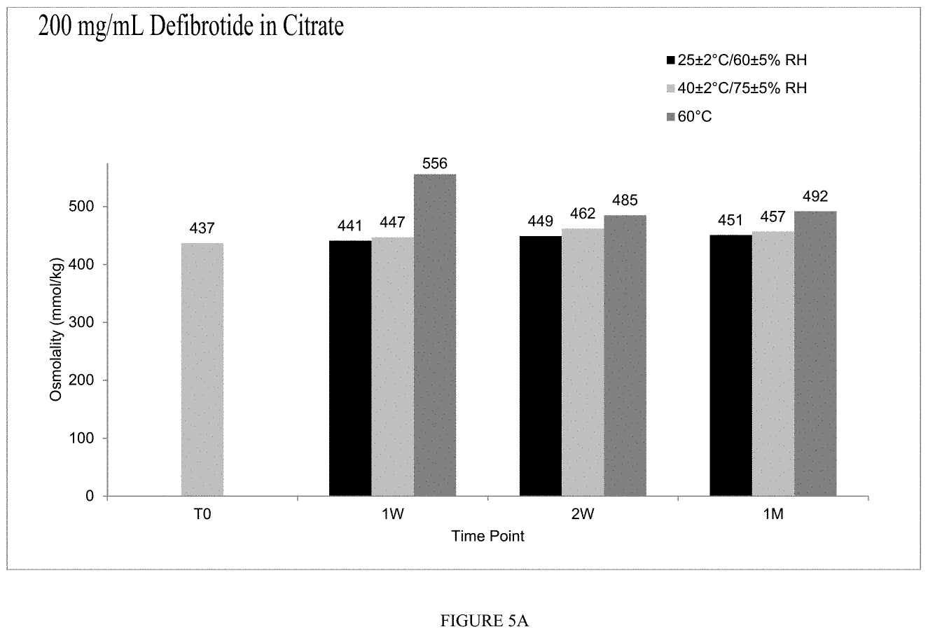

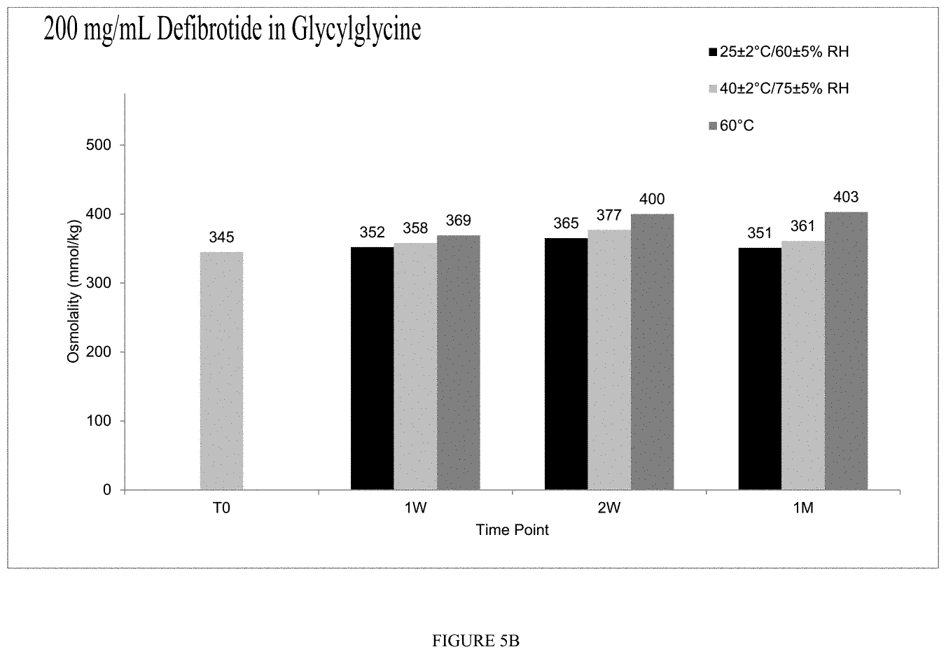

The graph in FIG. 5A shows the osmolality of 200 mg/mL defibrotide formulations using sodium citrate buffer.

The graph in FIG. 5B shows the osmolality of 200 mg/mL defibrotide formulations using Gly-Gly buffer. As seen in these graphs, the osmolality of the Gly-Gly formulations are reduced in comparison to formulations with citrate buffer. Importantly, the osmolality of the Gly-Gly formulations remains consistently low (below about 400 mOsm/kg) over each time point and at every temperature.

### 6.5 Example 5—Physical Stability and Product Profile Under Forced Degradation

The physical stability and product profile of HCLFs of the invention were evaluated using forced degradation studies. Defibrotide within the concentration range of 180 mg/mL and 220 mg/mL formulated in Gly-Gly and/or sodium citrate at pH 3 to pH 10, was evaluated after being stored at 25±2° C./60±5% relative humidity (“RH”), 40±2° C./75±5% RH and 60° C. for up to 3 months. Formulations tested are listed in Table 4 below.

TABLE 4
Summary of Formulations

| | Defibrotide | | | |
|---|---|---|---|---|
| Formula- | Conc. | | | |
| tion Code | (mg/mL) | Buffer Type | pH | Surfactant |
| FD1 | 80 | 34 mM Sodium | 7.3 | N/A |
| (Defitelio ® | | Citrate | | |
| Control) | | | | |
| FD2 | 200 | | | |
| FD3 | | | | |
| FD4 | 180 | 20 mM GlyGly | 7.5 | |
| | | | 3.0 | |
| FD5 | 200 | | 10.0 | |
| FD6 | | | | |
| FD7 | | 20 mM GlyGly, 10 | 7.3 | |
| | | mM Sodium Citrate | | |
| FD8 | | 34 mM GlyGly | 7.5 | 0.02% PS-80 |
| FD9 | 220 | 20 mM GlyGly | | N/A |

Based on the appearance, color, clarity, pH and particle count results from the forced degradation stability studies, defibrotide formulated at 200 mg/mL (FD3) in 20 mM Gly-Gly, pH between 7 and 8 was the most stable formulations following intended storage at 25±2° C./60±5% RH and stressed conditions for up to three months.

### 6.6 Example 6—Pharmacokinetics of Nucleic Acid Formulations Using Various Routes of Administration

### 6.6.1 Example 6.1—Intravenous (IV) Infusion, IV Bolus Injection, Subcutaneous (SC) Injection, and Intramuscular (IM) Injection of Defibrotide

The pharmacokinetics (PK) of various defibrotide formulations when administered via a single 2-hr intravenous (IV) infusion, IV bolus injection, subcutaneous (SC) injection, intramuscular (IM) injection, or oral (PO) gavage dose to male Gottingen pigs were compared. In addition, bioavailability of the various extravascular routes of administration was determined relative to IV infusion.

Male Gottingen pigs, or Minipigs, are the industry standard for exploring SC delivery, is an acceptable model for exploring defibrotide SC formulation and delivery options. Each animal received a single administration of defibrotide as listed in the treatment groups in Table 5. Gottingen pigs, n=3 males per group, were assigned to the treatment groups as shown:

TABLE 5
Study Design

| | |
|---|---|
| | Dose |
| Defibro- | Concen- |

| Group | | tide Dose | tration | Nominal Plasma Points PK |
|---|---|---|---|---|
| No. | ROA | (mg/kg) | (mg/mL)^{a} | Sampling Time |
| 1 | 2-hr IV | 25 | 4 | predose, 0.25, 0.5, 1, 2, 2.083, |
| | infusion | | | 2.25, 2.5, 3, 4, 6, 8, 12, and 24 |
| | | | | hr post-start of infusion |
| 2 | IV bolus | 2.5 | 2.5 | predose, 0.03, 0.083, 0.25, 0.5, 1, |
| | | | | 2, 3, 4, 5, 6, 8, 12, and 24 hr |
| | | | | postdose |
| 3 | SC | 25 | 80 | predose, 0.25, 0.5, 1, 1.5, 2, 3, 4, |
| 4 | IM | 25 | 80 | 5, 6, 8, 10, 12, and 24 hr postdose |
| 5 | PO | 100 | 80 | |

^{a}The dose volumes were 6.25, 1.0, 0.3125, 0.3125, and 1.25 mL/kg for Groups 1 through 5, respectively

Blood samples were collected and processed to plasma. A Quant-iT OliGreen ssDNA assay kit (Life Technologies) was used to quantify the concentration of defibrotide in pig plasma samples. Briefly, the assay methodology involves aliquoting the sample (in duplicate) into a 96 well plate, the addition of the OliGreen reagent, incubation with stirring (5 min, protected from light), and direct fluorescence measurement (485 excitation, 515 nm cutoff, and 525 nm emission). Assay ranges were 2.5 to 60 μg/mL (high range) and 0.05 to 2.5 μg/mL (low range). The lower limit of quantitation (LLOQ) of the assay was 0.05 μg/mL.

Individual animal defibrotide plasma concentration versus time data were downloaded into WinNonlin Phoenix version 6.3 software (Pharsight, Cary, N.C.) for PK analyses. A noncompartmental IV infusion, IV bolus, or extravascular injection model was used as appropriate to determine the single-dose PK parameters for each animal. Nominal dose and sample collection times (see Table 5) were used in estimating the PK parameters. Background values (range=0.115 to 0.903 μg/mL) were observed at the pre-dose time point in all animals. Therefore, concentration values below 1 μg/mL were treated as <LLOQ and were not used in the analyses. The following parameters were estimated whenever possible:
Tmax Time to maximum observed concentration
Cmax Maximum observed concentration
AUC0-t Area under the concentration-time curve from time=0 to the time point with the last measurable concentration, estimated by the linear trapezoidal rule
MRT0-t Mean residence time from time=0 to the time point with the last measurable concentration
Cmax/D Maximum observed concentration divided by dose level
AUC0-t/D Area under the concentration-time curve from time=0 to the last measurable concentration, estimated by the linear trapezoidal rule divided by dose level
CL Calculated for the IV groups as dose divided by AUC0-t

The bioavailable fraction (F), expressed as a percentage, was calculated for each animal, relative to the IV infusion dose group, as follows based on AUC0-t/D values:

(individual animal SC, IM, or PO AUC0-t/D)/(group mean IV infusion AUC0-t/D)×100%

Defibrotide Plasma Analyses: summarized defibrotide plasma concentrations following IV, SC, IM, or PO dosing to male Gottingen pigs showed the following: after a single IV infusion administration of 25 mg/kg or a single IV bolus administration of 2.5 mg/kg defibrotide, mean plasma concentrations were above 1 μg/mL out to 8 hr post-dose. Following SC or IM administration of 25 mg/kg defibrotide, mean plasma concentrations were greater than 1 μg/mL out to 24 hr post-dose (the last measured time point). Following a 100 mg/kg PO dose, defibrotide plasma concentrations were greater than 1 μg/mL at one time point in one animal (4 hr post-dose in animal 14M) and at three time points in one animal (5, 6, and 12 hr post-dose in animal 13M), but were less than 1 μg/mL at all time points in the third animal (15M).

Pharmacokinetic Analyses: individual animal and summarized PK parameters were also measured and showed the following: after a 2-hr IV infusion of 25 mg/kg defibrotide, the mean Cmax/D was 1.52 (μg/mL)/(mg/kg) and the mean AUC0-t/D value was 3.56 (hr*μg/mL)/(mg/kg). Following IV bolus administration of 2.5 mg/kg defibrotide, the mean Cmax/D was 14.4 (μg/mL)/(mg/kg) and the mean AUC0-t/D value was 8.30 (hr*μg/mL)/(mg/kg).

The Tmax following SC administration of 25 mg/kg defibrotide ranged from 0.25 to 8 hr post-dose, although multiple peaks were observed in the plasma PK profiles. The mean SC bioavailability (% F) was 81.3%. The Tmax following IM administration of 25 mg/kg defibrotide ranged from 0.25 to 0.50 hr post-dose. The mean IM bioavailability was 108%. In contrast, there were very few measurable concentrations following oral administration of 100 mg/kg defibrotide. The mean bioavailability following PO administration was less than 7.2%.

These results show that exposure to defibrotide was prolonged after SC and IM administration, relative to IV administration. The mean MRT last values were 9.26 and 7.36 hr in the SC and IM dose groups, respectively, compared to 1.30 and 2.16 hr in the IV infusion and IV bolus dose groups, respectively.

### 6.6.2 Example 6.2—Subcutaneous Administration of HCLF Defibrotide Formulations

To further investigate the pharmacokinetics of subcutaneously administered high concentration liquid formulations of defibrotide, three different HCLF formulations at 200 mg/mL were compared to a single 2-hr intravenous (IV) infusion or SC injection of Defitelio. In addition, their bioavailability via SC routes of administration was determined relative to IV infusion.

The HCLF formulations at 200 mg/mL were prepared as described above using sodium citrate, Gly-Gly or a combination of these buffers as indicated. Defitelio was administered at 4 mg/mL IV or 80 mg/mL SC using the doses shown in Table 6 below. Male Gottingen pigs (n=3 males per group) received a single administration of the test article listed in Table 6. Defibrotide was analyzed using the analytical method in Example 6.1. The PK parameters were determined similarly as in Example 6.1.

TABLE 6
Administration of Various Formulations

| | Route/Test | Dose | Concentration | Dose Volume |
|---|---|---|---|---|
| Group | Article | (mg/kg) | (mg/mL) | (mL/kg) |
| 1 | 2-hr IV (Defitelio) | 25 | 4 | 6.25 |
| 2 | SC (Defitelio) | 25 | 80 | 0.3125 |
| 3 | SC (HCLF-1) | 25 | 200 | 0.125 |
| 4 | SC (HCLF-2) | 25 | 200 | 0.125 |
| 5 | SC (HCLF-3) | 25 | 200 | 0.125 |

Note:
HCLF1: 34 mM sodium citrate, pH 7.3; HCLF2: 20 mM GlyGly, pH 7.3; HCLF3: 20 mM GlyGly, 10 mM sodium citrate, pH 7.3

The bioavailability expressed as a percent (% F) was calculated as reported above and the results are shown in Table 7 below.

TABLE 7
PK parameters of Defibrotide following SC and IV Administration

| | Cₘₐₓ | | AUC₀₋ₜ | MRT₀₋ₜ | |
|---|---|---|---|---|---|
| Treatment | μg/mL | Tₘₐₓ h | μg.h/mL | h | F % |
| IV, 2-h infusion | 44.8 (5%) | 0.500 | 91.6 | 2.41 | N/A |
| | | (0.250-1.00) | (4%) | (64%) | |
| SC, Defitelio | 7.03 | 0.500 | 56.1 | 9.28 | 61.3% |
| (80 mg/mL) | (17%) | (0.250-0.500) | (16%) | (7.6%) | (16%) |
| SC HCLF1 | 6.40 | 5.00 | 67.2 | 9.91 | 73.4% |
| (200 mg/mL) | (26%) | (4.00-12.0) | (42%) | (3%) | (42%) |
| SC HCLF2 | 7.45 | 1.00 | 59.8 | 10.9 | 65.3% |
| (200 mg/mL) | (62%) | (0.500-12.0) | (30%) | (20%) | (30%) |
| SC HCLF3 | 5.80 | 2.00 | 45.7 | 9.47 | 49.9% |
| (200 mg/mL) | (45%) | (1.00-4.00) | (24%) | (19%) | (24%) |

Mean and % CV values reported except for Tₘₐₓ, for which median and range of observed values (minimum-maximum) are reported.
F (bioavailability): calculated as AUC₀₋ₜ with SC dosing divided by the geometric mean AUC₀₋ₜ for the IV treatment
N/A: not applicable

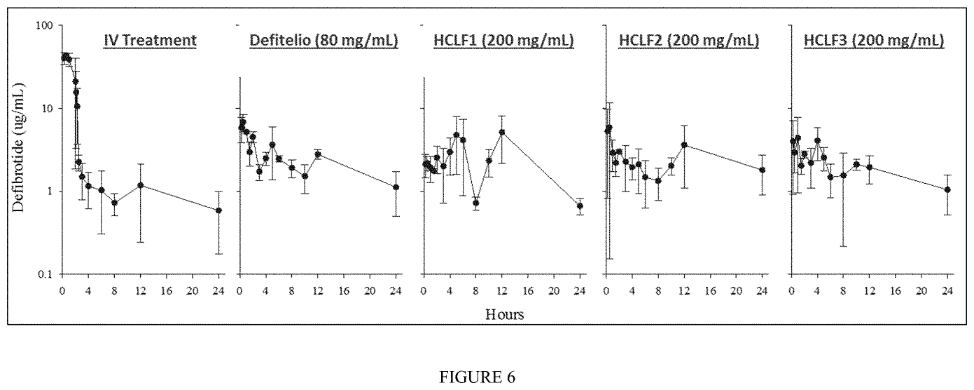

Plasma concentrations of defibrotide and plasma concentration-time data were determined as above and are shown in FIG. 6. The PK profiles in individual minipigs seen in FIG. 6 show multiple absorption peaks for all four SC treatments (Defitelio and the 3 HCLFs). As defibrotide is a mixture of oligonucleotides, the multiple peaks may be due to variation in the rates of absorption of the individual components of defibrotide. Taken together, the results indicate that bioavailability of defibrotide is favorable with SC dosing across all formulations, including the high concentration liquid formulations.

In addition, the mean residence times (MRT) of four SC groups ranged from 9.28-10.9 hours; while MRT of the IV group was just 2.41 hours (Table 7); thus the SC administration provided sustained release of defibrotide at approximately four and half times that of the IV infusion. This is consistent with what was shown in Example 6.1, in that SC administration of defibrotide in low-viscosity, HCLFs prolonged the plasma exposure of defibrotide in comparison to IV administration.

Though not wishing to be bound by any one theory, the extended circulation time by SC route is likely due to the nature of defibrotide HCLFs, which render a sustained release pattern of absorption. The extended circulation of defibrotide by SC administration of HCLFs may present an opportunity to investigate alternate regimens with less frequent dosing and improve quality of life for patients.

### 6.6.3 Example 6.3—Comparison of Pharmacokinetic Profiles of IV and SC Administrations

In an effort to demonstrate the PK comparison of SC HCLF administration, simulations of PK profiles following SC and IV infusion were conducted using the compartmental modeling techniques. A simple 1-compartmental model with or without a first-order absorption process was used to simulate the IV infusion or SC administration PK profile; respectively. In this modeling exercise, mean PK parameters following an IV infusion administration were taken from the package insert of Defitelio®. For PK studies, the clearance and volume of distribution of a drug are typically reported as a function of bioavailability; these are the CL/F and V/F, respectively. For the PK simulation used here, following SC HCLF administration the mean CL/F and V/F were calculated from the IV parameters by assuming a bioavailability of 70%. The absorption rate constant was assumed to be 0.22 h⁻¹, which is similar to that observed in Minipigs. The dose and regimen for the IV infusion were 6.25 mg/kg/infusion (a total daily dose of 25 mg/kg/day), by a 2-hour IV infusion, 4 times a day. The daily dose and regimen for a SC administration was 18 mg/kg/SC administration (a total daily dose of 36 mg/kg/day), 2 times a day. The simulation was conducted for a person with a body-weight of 70 kg. During the simulation, the total AUC following an SC administration was maintained to be the same as that of an IV infusion.

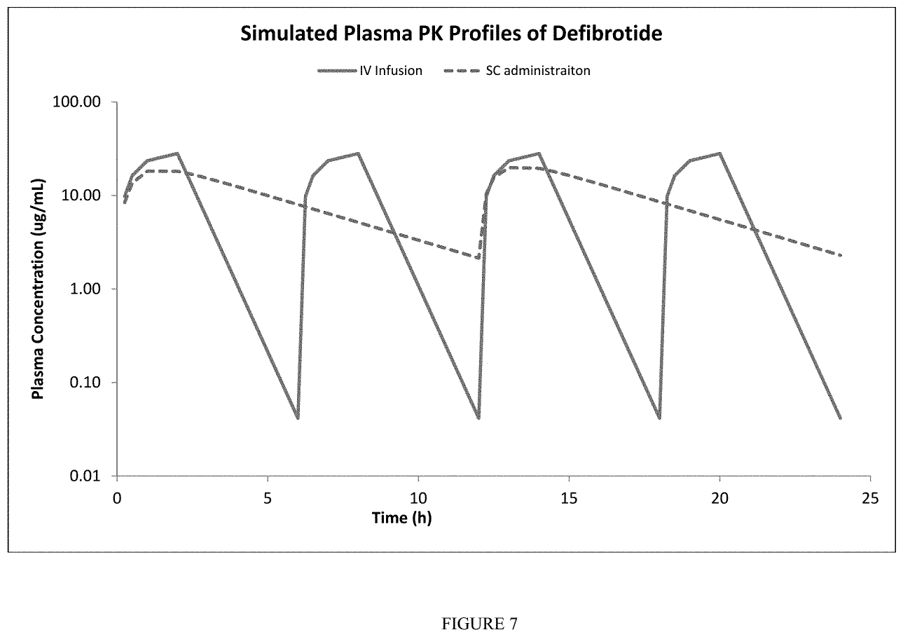

As shown in FIG. 7, the plasma concentration over time profiles demonstrate the slow, constant release of defibrotide following SC administration as opposed to the rapid clearance following each IV infusion. Importantly, the minimum plasma concentration of defibrotide following the SC administration was much higher than that of the IV infusion; while the Cmax of SC administration was similar to that of IV infusion.

The pharmacokinetics of the SC administration represents a profile that allows for continuous plasma exposure of defibrotide which may be important for its pharmacological activity. The peak-to-plasma concentration ratio following SC administration is about 8 as compared to that of the IV infusion which is about 700.

Together, these results demonstrate that subcutaneous administration of defibrotide provides a novel pharmacokinetic profile which differs significantly from the PK of IV infusions, such as those required by currently available defibrotide formulations. Slow and steady release of defibrotide may be critical for its benefit-to-risk profile and the unique subcutaneous pharmacokinetics allow for the development of new doses and/or dosing regimens which may yield better efficacy and improved safety profiles.

1-74. (canceled)

75. A pharmaceutical formulation comprising about 80 to about 100 mg/mL of defibrotide, and about 10 to about 40 mM sodium citrate, and wherein the formulation is formulated for subcutaneous delivery to a patient.

76. The pharmaceutical formulation of claim 75, wherein the formulation when administered subcutaneously demonstrates extended systemic half-life compared to the same defibrotide formulation administered intravenously.

77. The pharmaceutical formulation of claim 75, wherein the formulation exhibits lower peak-to-trough ratios of plasma concentrations compared to the same defibrotide formulation administered intravenously.

78. The pharmaceutical formulation of claim 75, wherein the formulation exhibits improved efficacy and/or an improved safety profile compared to the same defibrotide formulation administered intravenously.

79. The pharmaceutical formulation of claim 75, that is packaged for self-administration by a patient.

80. The pharmaceutical formulation of claim 75, wherein the formulation comprises about 34 mM sodium citrate.

81. The pharmaceutical formulation of claim 75, wherein the formulation comprises about 80 mg/mL defibrotide.

82. The pharmaceutical formulation of claim 75, wherein the formulation comprises about 80 mg/mL defibrotide and about 34 mM sodium citrate.

83. A device for subcutaneous administration of a formulation of claim 75.

84. The device of claim 83, wherein the formulation comprises about 80 mg/mL defibrotide and about 34 mM sodium citrate.

85. A method of treating or preventing a disease or condition comprising administering to a patient the formulation of claim 75, wherein the disease or condition is selected from thrombosis, Hematopoietic Stem Cell Transplantation (HSCT) related complications including sinusoidal obstruction syndrome or hepatic vaso-occlusive disease (VOD), Graft versus Host Disease (GvHD), Transplant-Associated Thrombotic Microangiopathy (TA-TMA) or Idiopathic Pneumonia Syndrome, other TMAs including Thrombotic Thrombocytopenic Purpura (TTP) and Hemolytic-Uremic Syndrome (HUS), Acute Myocardial Ischemia, Ischemic Stroke, Ischemia Reperfusion Injury, cytokine release syndrome (CRS) or Chimeric Antigen Receptor (CAR)-T Cell Related Encephalopathy Syndrome (CRES), Acute Respiratory Distress Syndrome (ARDS), Sickle Cell Vaso-occlusive Crisis (VOC), Sickle Cell Related Acute Chest Syndrome, Disseminated Intravascular Coagulation (DIC), Sepsis, Renal Insufficiency, other Coronary or Peripheral Artery Diseases, Hematological Malignancies or Solid Tumors.

86. The method of claim 85, wherein the formulation is administered at a dosing regimen that provides improved patient quality of life by requiring a reduced administration volume and/or allowing less-frequent administration and/or a shorter duration of administration.

87. The method of claim 85, wherein the formulation is self-administered by the patient using a device for subcutaneous administration.

88. The method of claim 87, wherein the formulation comprises about 80 mg/mL defibrotide and about 34 mM sodium citrate.

89. A method of treating or preventing Acute Respiratory Distress Syndrome comprising administering to a patient a pharmaceutical formulation comprising about 80 to about 100 mg/mL of defibrotide.

90. The method of claim 89, wherein the formulation comprises about 80 mg/mL defibrotide.

91. The method of claim 89, wherein the formulation comprises about 10 to about 40 mM sodium citrate.

92. The pharmaceutical formulation of claim 91, wherein the formulation comprises about 80 mg/mL defibrotide and about 34 mM sodium citrate.
